SEQUENCE LISTING (7 sequences)

SEQ ID NO: 1 - protein, 24 aa, synthetic construct
HHHHHHPMSDYDIPTTENLYFQGA

SEQ ID NO: 2 - protein, 105 aa, synthetic construct
HHHHHHPMSDYDIPTTENLYFQGANDHINLKVAGQDGSVVQFKIKRHTPLSKLMKAYCERQGLSMRQIRFRFDGQPINETDTPAQLEMEDEDTIDVFQQQTGGVY

SEQ ID NO: 3 - protein, 95 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVY

SEQ ID NO: 4 - protein, 119 aa, synthetic construct
HHHHHHPMSDYDIPTTENLYFQGAMADEKPKEGVKTENNDHINLKVAGQDGSVVQFKIKRHTPLSKLMKAYCERQGLSMRQIRFRFDGQPINETDTPAQLEMEDEDTIDVFQQQTGGVY

SEQ ID NO: 5 - protein, 119 aa, synthetic construct
HHHHHHPMSDYDIPTTENLYFQGAMADEKPKEGVKTENNDHINLKVAGQDGSVVQFKIKRHTPLSKLMKAYCERQGLSMRQIRFRFDGQPINETDTPAQLEMEDEDTIDVFQQQTAAVY

SEQ ID NO: 6 - protein, 4 aa, synthetic construct
GGVY

SEQ ID NO: 7 - protein, 4 aa, synthetic construct
AAVY

(19) United States
(12) Patent Application Publication
(10) Pub. No.: US 2026/0132176 A1
(43) Pub. Date: May 14, 2026

(54) COMPOSITIONS AND METHODS FOR TREATING NEURODEGENERATIVE DISEASE

(71) Applicants: The Trustees of Columbia University in the City of New York, New York, NY (US); The Governing Council of the University of Toronto, Toronto (CA)

(72) Inventors: Ottavio ARANCIO, New York, NY (US); Luana FIORITI, New York, NY (US); Paul Edward FRASER, Toronto (CA)

(21) Appl. No.: 19/329,433

(22) Filed: Sep. 15, 2025

Related U.S. Application Data
(63) Continuation-in-part of application No. PCT/US2024/020014, filed on Mar. 14, 2024.
(60) Provisional application No. 63/490,204, filed on Mar. 14, 2023.

(51) Int. Cl. C07K 14/47; A61K 38/00; A61P 25/28

(52) U.S. Cl. CPC C07K 14/47; A61P 25/28; A61K 38/00

(57) ABSTRACT

Methods and compositions for treating or preventing a neurodegenerative disease, such as Alzheimer's Disease, in a subject by administering to a subject a therapeutic amount of a pharmaceutical composition comprising a recombinant SUMO2 analogue.

### CROSS REFERENCE TO RELATED APPLICATION

This application is a continuation-in-part of International Patent Application No. PCT/US2024/020014, filed on Mar. 14, 2024, which claims the benefit of and priority to U.S. Provisional Application No. 63/490,204, filed on Mar. 14, 2023, the contents of each are hereby incorporated by reference their entireties.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### STATEMENT REGARDING FEDERALLY SPONSORED RESEARCH OR DEVELOPMENT

This invention was made with government support under Grant No. NS110024 awarded by the National Institute of Health. The Government has certain rights in the invention.

### REFERENCE TO SEQUENCE LISTING

This application incorporates-by-reference nucleotide and/or amino acid sequences which are present in the file named “93597-7607_92657-A-PCT-A_Sequence_Listing_AWG.xml”, which is 7,570 bytes in size, and which was created on Jan. 22, 2026 in the IBM-PC machine format, having an operating system compatibility with MS-Windows, which is contained in the XML file filed Jan. 23, 2026 as part of this application.

### BACKGROUND

Alzheimer's disease (AD) is characterized by the accumulation of amyloid-β (Aβ) peptides in the brain and affects more than 35 million people worldwide. Soluble Aβ accumulation in neurons results in synapse loss, which is associated with cognitive decline seen in patients with AD. A second neurological feature of AD is memory loss, which is associated with an impairment in long-term potentiation (LTP), a type of synaptic plasticity underlying memory formation. Therapeutic strategies against Alzheimer's disease (AD) have focused on enhancing the clearance of amyloid pathology and reducing the production and/or aggregation of Aβ. Symptomatic treatments have limited efficacy and it would be desirable to have therapies capable of limiting synaptic loss and dysfunction to reduce and possibly reverse the cognitive decline in AD.

Synucleinopathies like Parkinson's disease, Dementia with Lewy Bodies (DLB), and Multiple System Atrophy (MSA) are diagnosed in over 2.5 million people in the US, with around 180,000 new cases each year. The economic burden on our health system and on caregivers is expected to increase over the next 20 years in proportion to our aging population. Disease modifying therapies are urgently needed, particularly ones that can reduce pathology, prevent synaptic damage and improve motor function.

### SUMMARY

The present application is directed to compositions and methods for treating neurodegenerative diseases, such as Alzheimer's disease and Parkinson's disease.

In accordance with one aspect, a method for treating a neurodegenerative disease, such as Alzheimer's Disease or tauopathies, in a subject is disclosed. The method includes administering to a subject a therapeutic amount of a pharmaceutical composition comprising a recombinant small ubiquitin modifier 2 (SUMO2) analogue.

In accordance with another aspect, a method for increasing memory retention in a subject afflicted with a neurodegenerative disease by administering to the subject a therapeutic amount of a pharmaceutical composition comprising recombinant SUMO2 analogue is disclosed.

In accordance with yet another aspect, a method for increasing synaptic plasticity in a subject afflicted with a neurodegenerative disease by administering to a subject a therapeutic amount of a pharmaceutical composition comprising recombinant SUMO2 analogue is disclosed. In some embodiments, the synaptic plasticity comprises learning, memory, or a combination thereof. In some embodiments, the synaptic plasticity comprises long term potentiation (LTP).

In accordance with another aspect, a method for preventing Alzheimer's Disease in a subject by administering to a subject a therapeutic amount of a pharmaceutical composition comprising a recombinant SUMO2 analogue is disclosed.

In accordance with certain embodiments, the recombinant SUMO2 analogue includes a sequence that is at least 70% identical to a sequence of the SUMO2.

In accordance with certain embodiments, the recombinant SUMO2 analogue includes an N-terminal tag and a leader sequence N-terminal to the SUMO2 sequence.

In accordance with some embodiments, the recombinant SUMO2 analogue includes a SUMO2 sequence with a hexa-His tag and a leader sequence (HHHHHHPMSDYDIPTTENLYFQGA (SEQ ID NO: 1)) with a TEV cleavage site (GA) immediately N-terminal to SUMO2 sequence.

In accordance with certain embodiments, the recombinant SUMO2 analogue has the sequence

(SEQ ID NO: 2)
HHHHHHPMSDYDIPTTENLYFQGANDHINLKVAGQDGSVVQFKIKRHTP
LSKLMKAYCERQGLSMRQIRFRFDGQPINETDTPAQLEMEDEDTIDVFQ
QQTGGVY.

In accordance with one aspect, a pharmaceutical composition comprising a recombinant SUMO2 analogue is disclosed.

In accordance with certain embodiments, the pharmaceutical composition includes a recombinant SUMO2 analogue having a sequence that is at least 70% identical to a sequence of the SUMO2.

In accordance with certain embodiments, the pharmaceutical composition includes a recombinant SUMO2 analogue, which contains a hexa-His tag and a leader sequence (HHHHHHPMSDYDIPTTENLYFQGA (SEQ ID NO: 1)) with a TEV cleavage site (GA) immediately N-terminal to SUMO2 sequence.

In accordance with certain embodiments, the pharmaceutical composition includes a recombinant SUMO2 analogue with the sequence

(SEQ ID NO: 2)
HHHHHHPMSDYDIPTTENLYFQGANDHINLKVAGQDGSVVQFKIKRHTP
LSKLMKAYCERQGLSMRQIRFRFDGQPINETDTPAQLEMEDEDTIDVFQ
QQTGGVY.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A provides immunoblot analysis of transgenic mice expressing human SUMO2 by the prion cos-tet promoter within different brain regions (Ob—olfactory bulb; Cx—cortex; Cb—cerebellum; Hip—hippocampus).

FIG. 1B shows that SUMO2 overexpression protected against LTP impairment of APP mice.

FIG. 1C shows that contextual fear conditioning as a measure of associative memory impairments in APP mice were prevented by SUMO2 overexpression.

FIG. 1D shows quantification of soluble and insoluble Aβ42 as determined by sandwich ELISA.

FIG. 1E shows immunoblotting for SUMO2 in SUMO2-APP transgenics as compared to APP mice only.

FIG. 2A provides a schematic of the prophylactic therapy time course for treatments with SUMO2 biologics, BioSenA and BioSenI (20 mg/kg; SC. 3×/wk) and saline controls.

FIG. 2B is a graph showing LTP test results under different conditions.

FIG. 2C is a graph showing associative memory as determined by contextual fear conditioning under different conditions.

FIG. 2D shows ELISA quantification of soluble and insoluble Aβ42 in cortex-hippocampal homogenates under different conditions.

FIG. 2E provides timelines for a Reversal study with BioSenA/BioSenI and treatment of APP transgenics with pre-existing amyloid pathology and LTP/cognitive impairments at 6-months (moderate AD) until 9-months of age (severe AD).

FIG. 2F is a graph showing LTP test results under different conditions.

FIG. 2G is a graph showing associative memory as determined by contextual fear conditioning under different conditions.

FIG. 2H shows SUMO2 immunoblotting for whole brain homogenates from BioSenA-treated APP transgenics relative to BioSenI or Saline treated mice.

FIG. 3A a graph showing LTP test results under different conditions.

FIG. 3B is a graph showing associative memory as determined by contextual fear conditioning under different conditions.

FIG. 3C is a bar chart providing a sensory threshold assessment under different conditions.

FIG. 4A provides representative images showing amyloid immunohistochemistry in the APP and SUMO2-APP transgenics.

FIG. 4B provides quantitative image analysis for dense and diffuse amyloid plaques revealed comparable levels in APP and SUMO2-APP transgenic mice.

FIG. 4C provides image results for amyloid processing in APP and SUMO2-APP transgenics.

FIG. 4D shows ELISA assays for the secreted APP β-fragment (sAPPbeta-sw) at 6- and 9-months.

FIG. 4E provides bar charts for endogenous murine Aβ42 in SUMO2 over-expressing mice relative to Non-Tg animals (6-months of age).

FIG. 5A is a graph showing BioSenA concentration in plasma after intravenous injection.

FIG. 5B is a graph showing BioSenA concentration in plasma after subcutaneous injection.

FIG. 5C is a graph showing BioSenA concentration in the cortex after intravenous injection.

FIG. 5D is a graph showing BioSenA concentration in the cortex after subcutaneous injection.

FIG. 6A provides immunocytochemistry images for the prophylactic treatments with BioSenA, BioSenI or Saline.

FIG. 6B provides immunocytochemistry images for the reversal treatments with BioSenA, BioSenI or Saline.

FIG. 6C provides a bar chart showing dense plaque cores and diffuse halos for APP-Tg mice treated prophylactically at the pre-pathology and cognitively normal stages.

FIG. 6D provides a bar chart showing dense plaque cores and diffuse halos for APP-Tg mice treated after amyloid pathology and cognitive impairments were well-established.

FIG. 7A provides bar charts showing soluble Aβ42 levels in homogenates from dissected cortex, olfactory bulb and cerebellum as determined by sandwich ELISA.

FIG. 7B provides bar charts showing insoluble Aβ42 levels in homogenates from dissected cortex, olfactory bulb and cerebellum as determined by sandwich ELISA.

FIG. 8A provides immunoblotting of whole brain homogenates from the prophylactic treated with BioSenA as compared to saline treated APP animals.

FIG. 8B provides quantification of SUMO2 conjugates in brain homogenates from the reversal study.

FIG. 8C provides quantification of monomeric free SUMO2 in brain homogenates from the reversal study.

FIG. 8D shows immunoblot for PSD95 from brain homogenates (cortex-hippocampus combined) in BioSenA treated mice as compared to BioSenI or Saline.

FIG. 8E provides bar charts for quantification of PSD95 immunoreactivity in BioSenA treated mice as compared to BioSenI or Saline.

FIG. 9 is a graph showing LTP test results under different conditions.

FIG. 10 is a graph showing LTP test results under different conditions.

FIG. 11 provides photographs of immunohistochemical staining for the phosphorylation at Serine-129.

FIG. 12 shows graphs of Rotarod and Vertical Screen Tests for BioSenA treated mice.

FIG. 13 provides images illustrating immunofluorescence analysis of α-synuclein pathology as measured by the levels of the phosphorylated Ser-129 residue (pS129; green).

FIGS. 14A and 14B provide photographs of immunohistochemical staining showing accumulation of pS129 asyn immunoreactivity progressively increased after 7-days (FIG. 14A) or 14-days (FIG. 14B).

FIGS. 15A and 15B provide photographs of immunohistochemical staining showing BioSenA treatment reduces asyn aggregate-induced accumulation and mislocalization of synaptic protein SV2A.

FIG. 16 provides photographs of immunohistochemical staining and graphs showing cells treated with BioSenA displayed much lower seeding capacity compared to untreated neurons.

FIG. 17 shows a schematic methodology for determining if SUMO2 prevents spreading of α-Syn pathology in vivo.

FIG. 18 provides photographs of immunohistochemical staining showing the hippocampus region from control non-transgenic mice and SUMO2 transgenics that were inoculated with a-synuclein preformed fibrils (PFFs).

FIG. 19 shows bar charts showing levels of phosphorylated a-synuclein (pS129) in the cortex and the hippocampus in non-transgenic (Non-Tg) or SUMO2 over-expressing transgenics (SUMO2).

### DETAILED DESCRIPTION

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount” or “therapeutically effective amount” as used herein is an amount of a compound that is sufficient to effect beneficial or desired results, including clinical results. As such, the effective amount may be sufficient, for example, to reduce or ameliorate the severity and/or duration of an affliction or condition, or one or more symptoms thereof, prevent the advancement of conditions related to an affliction or condition, prevent the recurrence, development, or onset of one or more symptoms associated with an affliction or condition, or enhance or otherwise improve the prophylactic or therapeutic effect(s) of another therapy. An effective amount also includes the amount of the compound that avoids or substantially attenuates undesirable side effects.

Small ubiquitin modifier (SUMO) proteins provide pivotal roles in synaptic biology. SUMOylation has been linked to AD and related neurodegenerative disorders including Huntington's and Parkinson's disease and the two major isoforms—SUMO1 and SUMO2—exhibit disparate effects on pathogenesis and disease progression likely due to isoform functional heterogeneity. SUMO1 is largely conjugated to target proteins under basal conditions and exacerbates amyloid pathology and leads to a reduction in dendritic spine densities. In contrast, SUMO2 conjugation is essential for long term potentiation (LTP), a type of synaptic plasticity thought to underlie memory formation, and hippocampal-dependent learning and provokes a neuroprotective response to stress such as transient ischemia. SUMO2 may represent a potential therapeutic avenue to counteract Ab-induced synaptotoxicity as well as learning and memory deficits in AD.

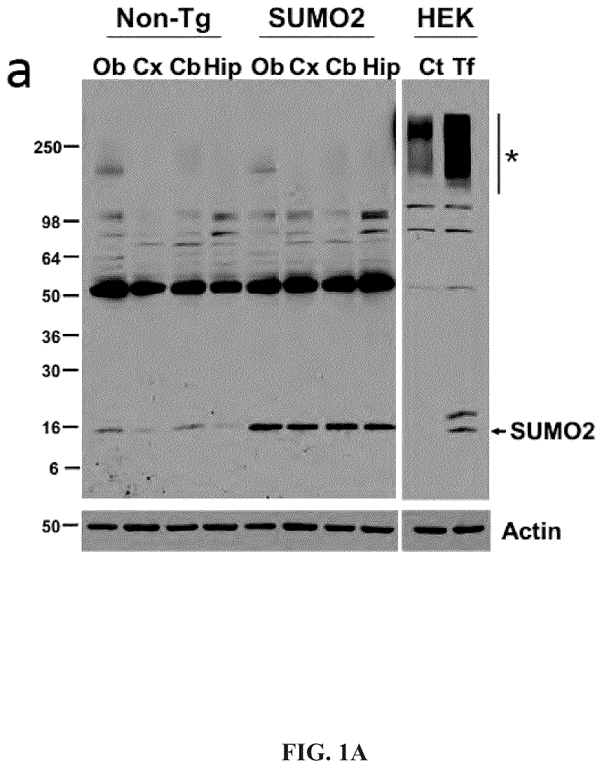

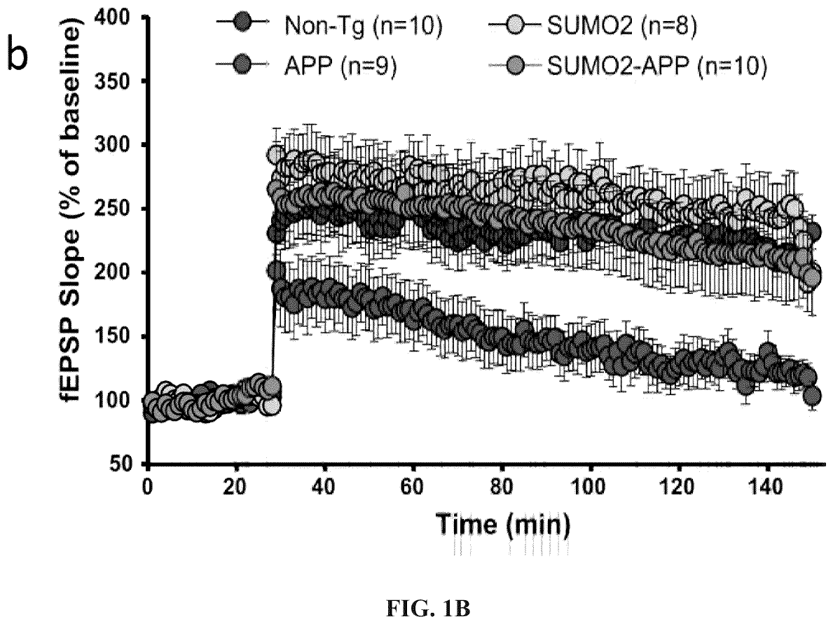

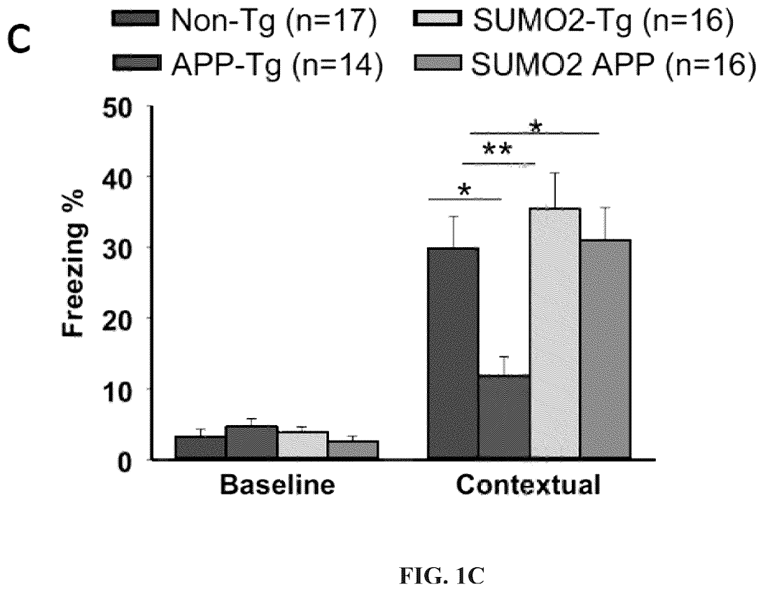

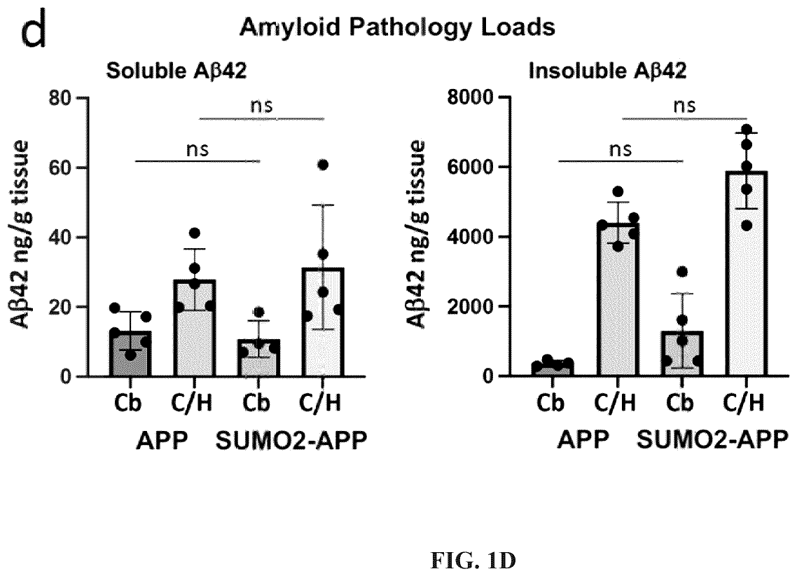

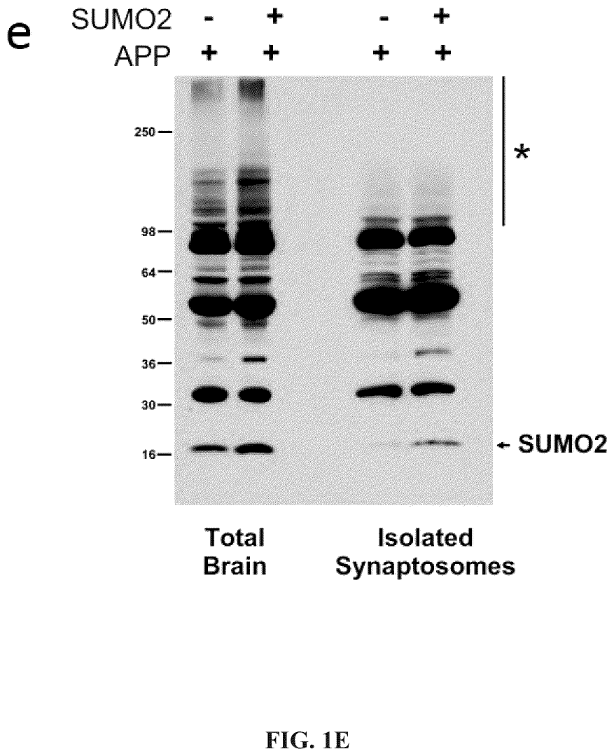

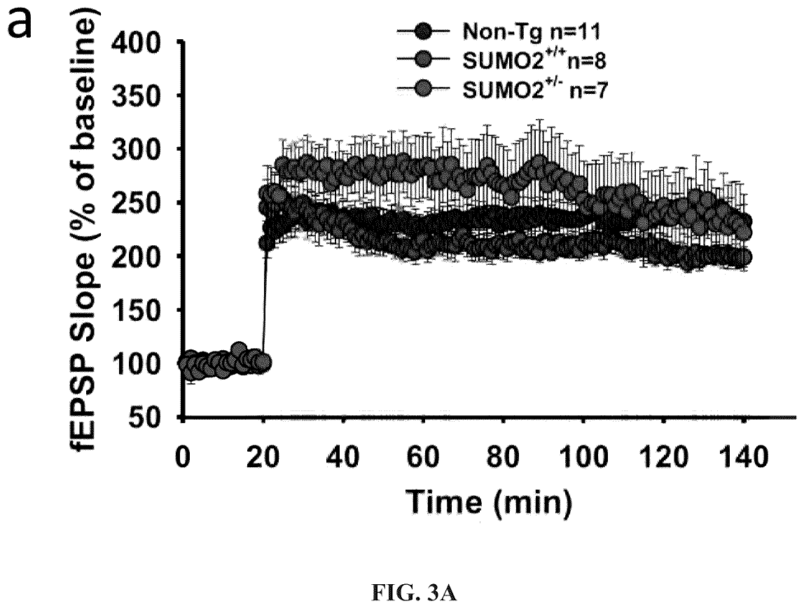

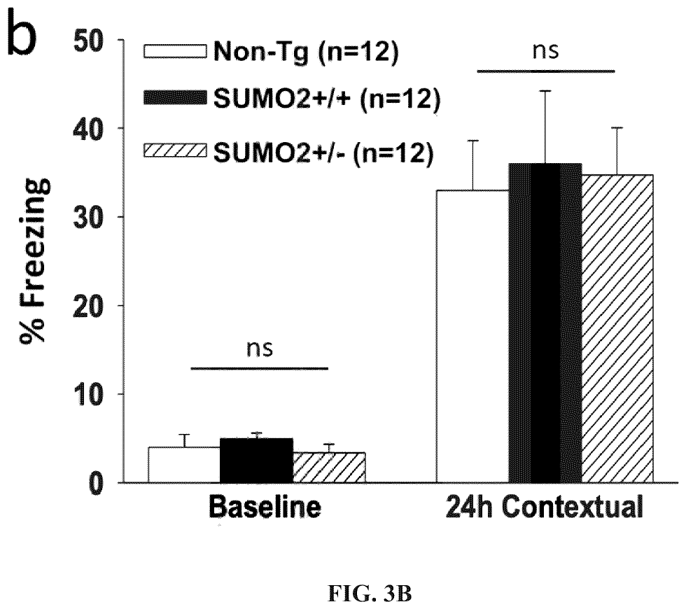

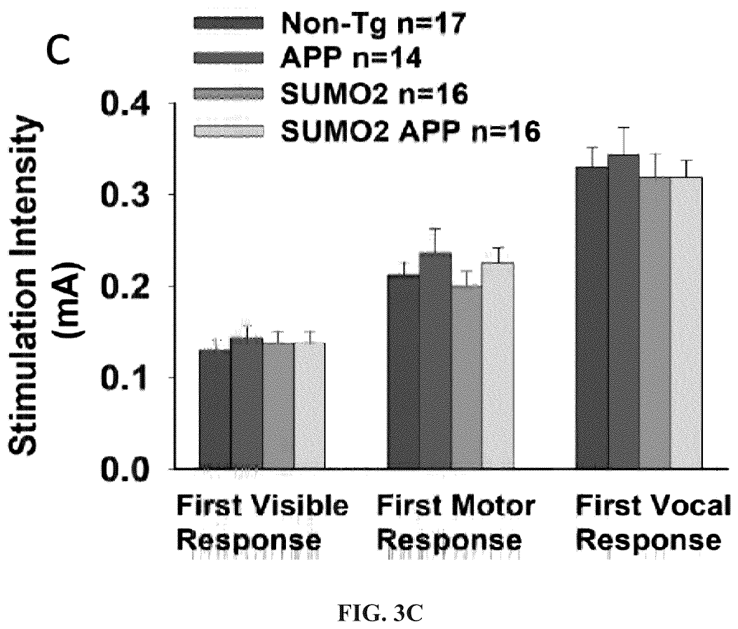

To assess its impact on AD pathology, neuronal SUMO2 was genetically elevated in a transgenic mouse model (SUMO2) with expression driven by the prion-cos-tet promoter. Elevated SUMO2 was observed throughout the brain and, under basal conditions, no substantial increases in higher molecular weight conjugates were observed (FIG. 1A). SUMO2 transgenic mice (6-months of age) displayed normal LTP and memory as determined by fear conditioning (See FIGS. 3A and 3B). Crossbreeding the SUMO2 with APP over-expressing mice (APP) generated double-transgenics (SUMO2-APP) which were examined at 9-months of age when amyloid plaque load is extensive and representative of late-stage disease. Increased SUMO2 prevented LTP impairment in SUMO2-APP transgenics as compared to APP mice (FIG. 1B).

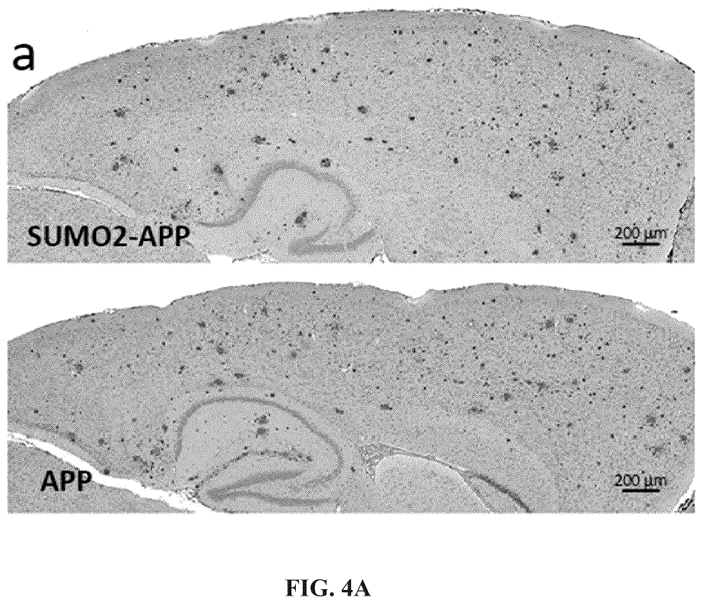

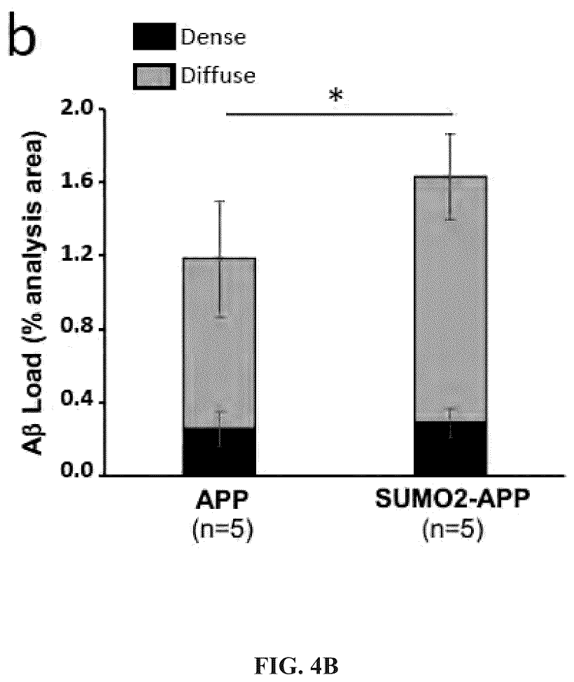

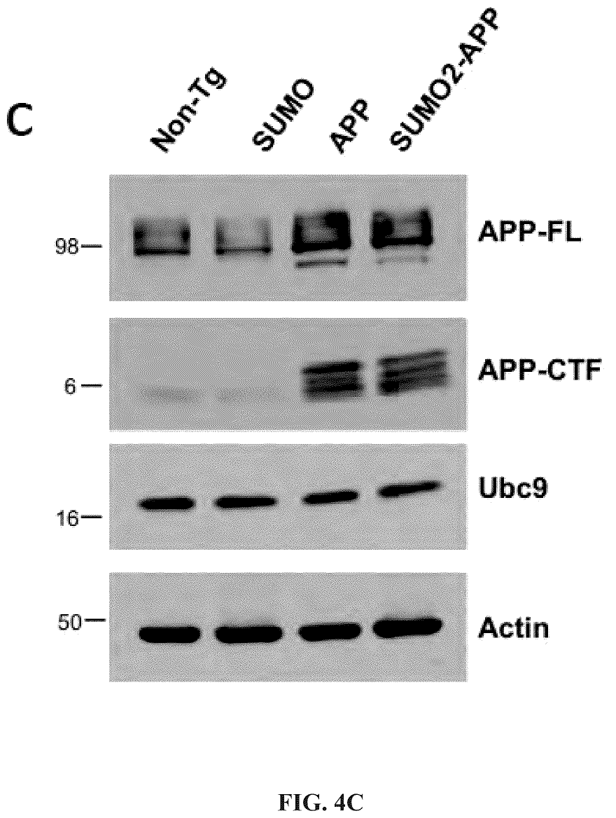

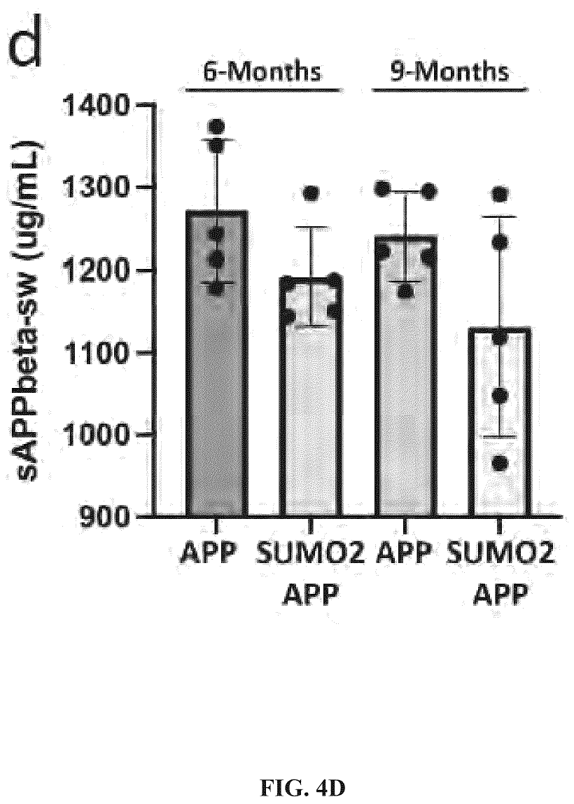

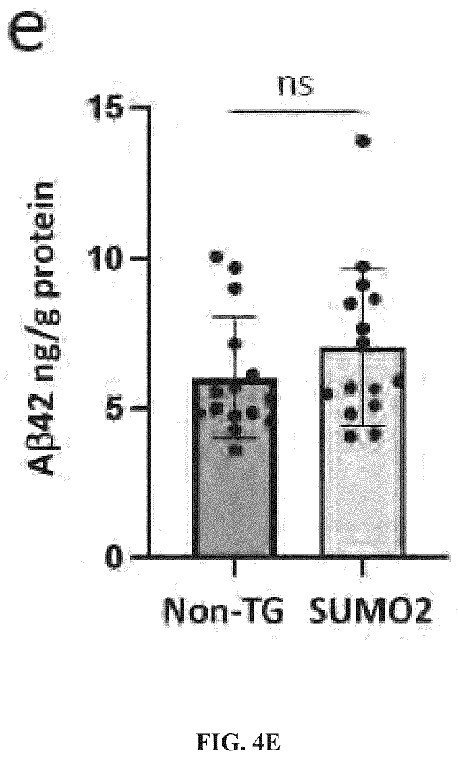

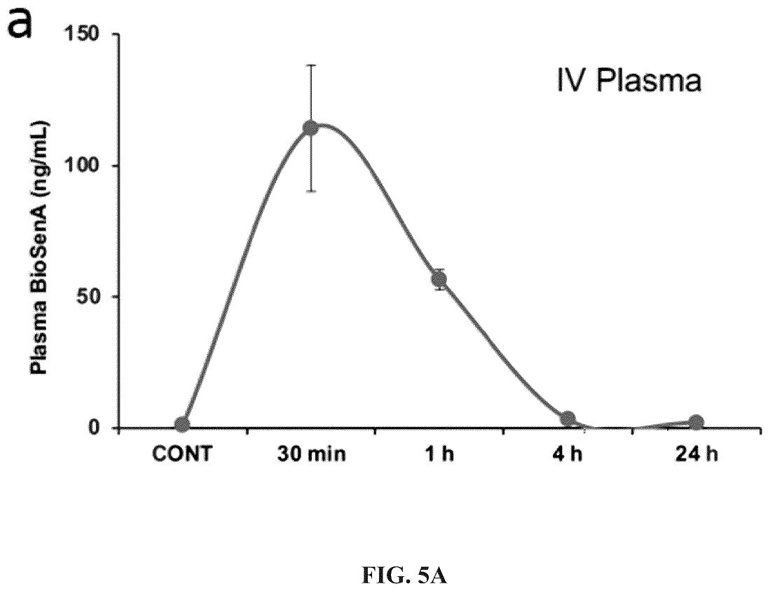

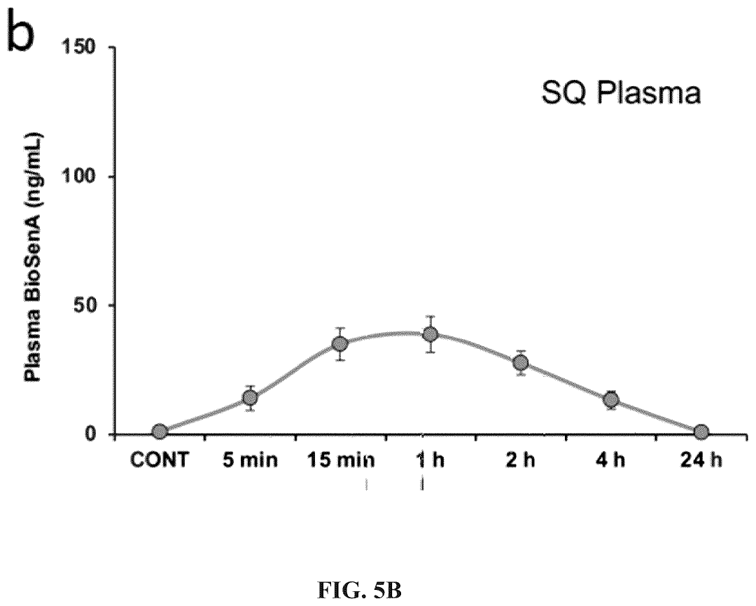

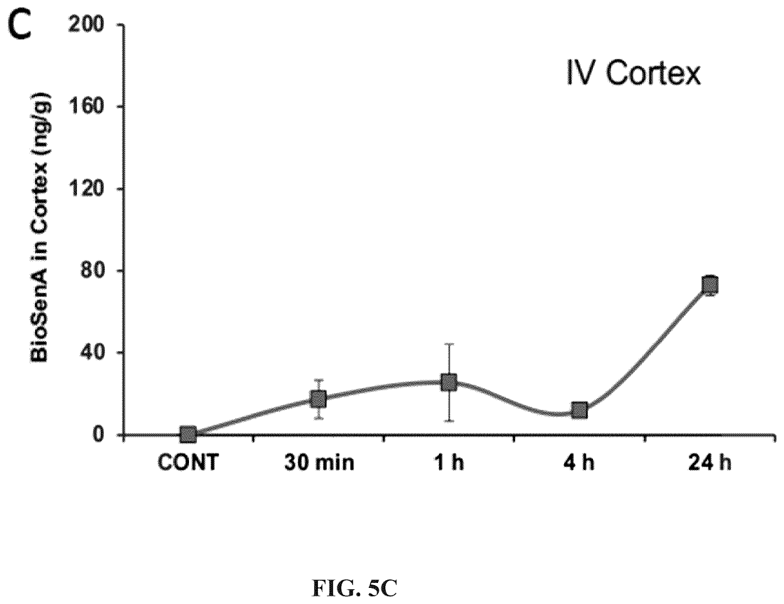

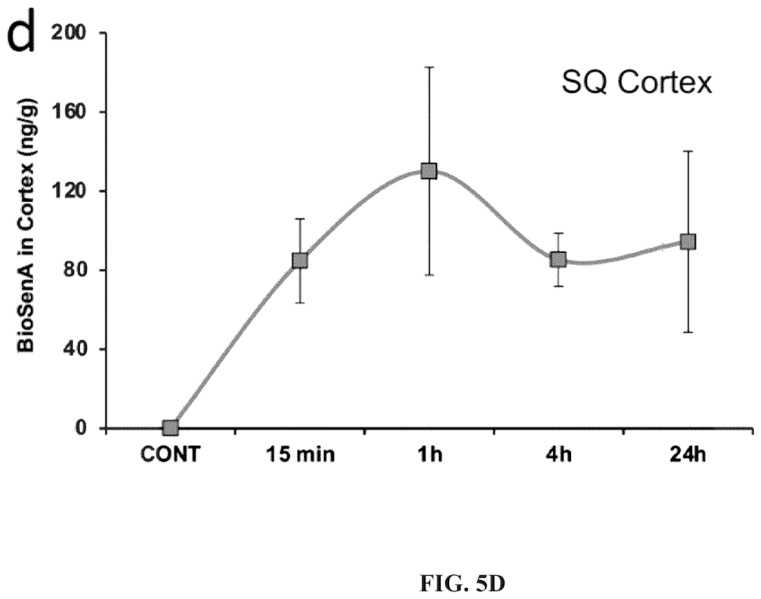

This effect was supported by fear memory analysis where SUMO2 mice were comparable to Non-Tg animals and APP transgenics exhibited the expected cognitive dysfunction whereas SUMO2-APP transgenics exhibited normal memory (FIG. 1C) with no changes in mouse ability to perceive the electric shock (FIG. 5C). SUMO2 does not act by reducing amyloid load since soluble and insoluble Aβ42 levels were comparable in APP and SUMO2-APP transgenics (FIG. 1D). Amyloid immunocytochemistry also indicated the typical distribution and expected density of plaques within the hippocampus and cortex of both the APP and SUMO2-APP transgenics (FIG. 4A). Quantification by image analysis of the Ab deposits revealed no differences in the dense plaques, however, the area of the diffuse halos surrounding plaques was slightly increased in the SUMO2-APP animals (FIG. 4B). In addition, SUMO2 over-expression had no effect on APP processing as APP C-terminal and b-secretase cleavage fragments were unchanged and endogenous murine Aβ levels were comparable in SUMO2 transgenic and Non-Tg animals (FIGS. 4C, 4D and 4E).

A potential mechanism of action is an increase in SUMO2-modified proteins as elevated levels of the high molecular weight conjugates are seen in the SUMO2-APP animals, and possibly a direct effect within synaptic proteins given the observed increase in free SUMO2 in isolated synaptosomes (FIG. 1E). These data indicate that SUMOylation is a significant regulator of neuronal pathways and the specific enhancement of SUMO2 levels and its subsequent conjugation may be beneficial for the treatment of AD. To this end, a recombinant biologic sentrin, BioSenA, was developed which mimics SUMO2 activity and its therapeutic efficacy was investigated in the model of AD amyloid pathology.

The term “SUMO2 recombinant analogue” as used herein, refers to a protein or an amino acid sequence that differs by one or more amino acids from the amino acid sequence of SUMO2. In certain embodiments, the present application provides SUMO2 recombinant analogues having a certain % identity to SUMO2 or to some other SUMO2 variant. The following terms are used to describe the sequence relationships between two or more polynucleotides or amino acid sequences: “sequence identity,” “percentage sequence identity” and “identity.” These terms are used in accordance with their usual meaning in the art. Percentage sequence identity is measured with reference to a reference sequence. The term “sequence identity” means that two polynucleotide or amino acid sequences are identical (i.e. on a nucleotide-by-nucleotide basis). The term “percentage of sequence identity” is calculated by comparing two optimally aligned sequences, determining the number of positions at which the identical nucleic acid base or amino acid occurs in both sequences to yield the number of matched positions, dividing the number of matched positions by the total number of positions, and multiplying the result by 100 to yield the percentage of sequence identity.

In one embodiment, the present application provides a SUMO2 recombinant analogue that comprises, consists essentially of, or consists of an amino acid sequence that is at least 70%, or at least 75%, or at least 80%, or at least 85%, or at least 90%, or at least 95%, or at least 98% identical to the sequence of the SUMO2. In accordance with some embodiments, the recombinant SUMO2 analogue comprises a SUMO2 sequence or portion thereof, with an N-terminal tag and a leader sequence N-terminal to the SUMO2 sequence or portion thereof. In accordance with another embodiment, the recombinant SUMO2 analogue comprises a SUMO2 sequence or portion thereof with a hexa-His tag and a leader sequence. In some embodiments, the hexa-His tag and a leader sequence has a sequence comprising (HHHHHHPMSDYDIPTTENLYFQGA (SEQ ID NO: 1)) with a TEV cleavage site (GA) immediately N-terminal to the SUMO2 sequence or portion thereof.

SUMO2 has the following sequence:

(SEQ ID NO: 3)
MADEKPKEGVKTENNDHINLKVAGQDGSVVQFKIKRHTPLSKLMKAYCE
RQGLSMRQIRFRFDGQPINETDTPAQLEMEDEDTIDVFQQQTGGVY.

BioSenA is a SUMO2 mimetic or SUMO2 recombinant analogue. In accordance with one embodiment, BioSenA is a SUMO2 mimetic comprising amino acid 13-95 plus His tag and leader sequence. In accordance with one embodiment, BioSenA comprises a SUMO2 recombinant analogue with a hexa-His tag and leader sequence with a cleavage site immediately N-terminal to the SUMO2 sequence. In accordance with one embodiment, BioSenA has a hexa-His tag and leader sequence (HHHHHHPMSDYDIPTTENLYFQGA (SEQ ID NO: 1)) with a TEV cleavage site (GA) immediately N-terminal to the SUMO2 sequence. The SUMO2 sequence can be the full sequence or a portion thereof. In accordance with one embodiment, the SUMO2 sequence corresponds to the full SUMO2 sequence and BioSenA has the following sequence:

(SEQ ID NO: 4)
HHHHHHPMSDYDIPTTENLYFQGAMADEKPKEGVKTENNDHINLKVAGQ
DGSVVQFKIKRHTPLSKLMKAYCERQGLSMRQIRFRFDGQPINETDTPA
QLEMEDEDTIDVFQQQTGGVY.

In accordance with one embodiment, the SUMO2 sequence corresponds to a partial SUMO2 sequence and BioSenA has the following sequence:

(SEQ ID NO: 2)
HHHHHHPMSDYDIPTTENLYFQGANDHINLKVAGQDGSVVQFKIKRHTP
LSKLMKAYCERQGLSMRQIRFRFDGQPINETDTPAQLEMEDEDTIDVFQ
QQTGGVY.

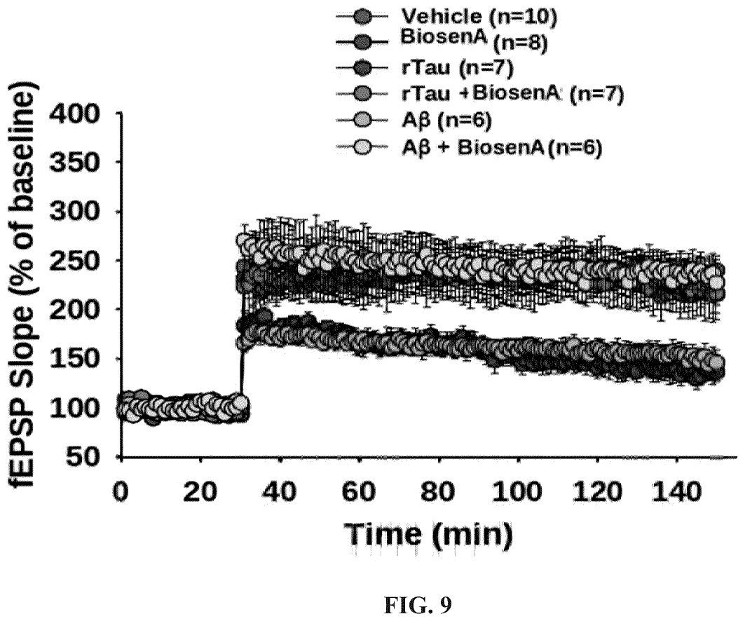

BioSenA with the full SUMO2 sequence was used in all testing disclosed herein except for test results presented in FIG. 9, where the partial SUMO2 sequence disclosed herein was used.

BioSenI is an inactive form of the SUMO2 mimetic having the following sequence:

(SEQ ID NO: 5)
HHHHHHPMSDYDIPTTENLYFQGAMADEKPKEGVKTENNDHINLKVAGQ
DGSVVQFKIKRHTPLSKLMKAYCERQGLSMRQIRFRFDGQPINETDTPA
QLEMEDEDTIDVFQQQTAAVY.

The manufacturing process for the recombinant SUMO2 analogues disclosed herein includes the use of lab grade, non-GLP production of recombinant proteins from E. Coli. Proteins were expressed in E. coli grown in Terrific Broth containing antibiotic (50 μg/ml kanamycin) with induction (1 mM IPTG) at 37° C. for 4 hours. Cell pellets were resuspended in buffer (20 mM HEPES, 300 mM NaCl, 20 mM imidazole; pH 7.4) and lysed by sonication. Lysates were clarified by centrifugation (10,000 g) and applied to a nickel affinity column (Qiagen Superflow). Bound protein was washed with buffer containing 40 mM imidazole and eluted with 300 mM imidazole. Eluates were pooled and buffer exchanged by dialysis to phosphate buffered saline with 1 mM dithiothreitol. Protein was concentrated using Amicon Ultra-15 with a molecular weight cut-off of 10 kD and purified by size exclusion column (Cytvia Superdex 75) in PBS. Fractions containing purified proteins were sterile filtered with Acrodisc units with Mustang E membrane and aliquots snap frozen in liquid nitrogen then stored at −80° C.

Distribution and bioavailability of BioSenA in the periphery and brain were determined by sandwich ELISA for a His-tagged version of the SUMO2 recombinant biologic. BioSenA levels following intravenous (IV) injection were maximal at ˜30 mins in plasma and no detectable levels were observed at 4 hours (FIG. 5A). In contrast, subcutaneous (SQ) injection resulted in a peak for BioSenA at ˜1 hr with levels that were still detectable at 4 hours (FIG. 5B). Brain levels for BioSenA by IV administration correlated with a slow increase over the course of 4 hours and was further elevated at 24 hr (FIG. 5C). Subcutaneous (SQ) administration was equally effective at brain penetration as significant levels were observed at 15 minutes that were comparable to 4 hours post injection (FIG. 5D). SQ administration also resulted in the maintenance of high BioSenA brain concentrations for up to 24 hours. BioSenA was found to have no adverse effects on body or tissue weight and liver and kidney enzymes were not elevated and pathological analysis of liver showed no differences for acute (300 mg/kg; 7 days) or chronic (100 mg/kg; 15 days) doses indicating a positive safety profile.

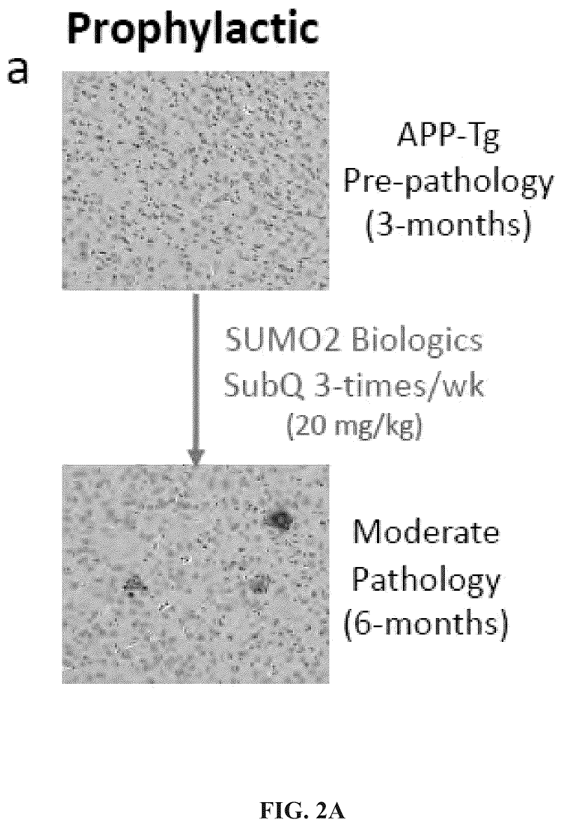

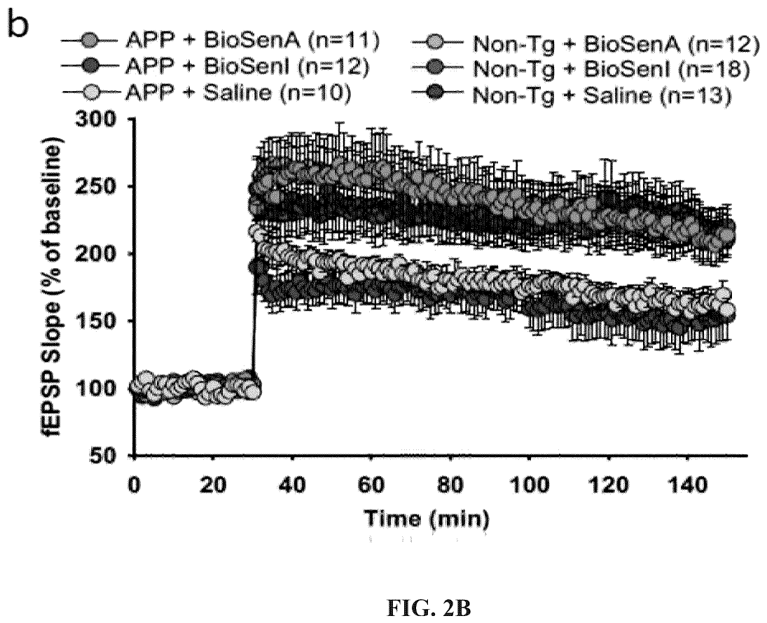

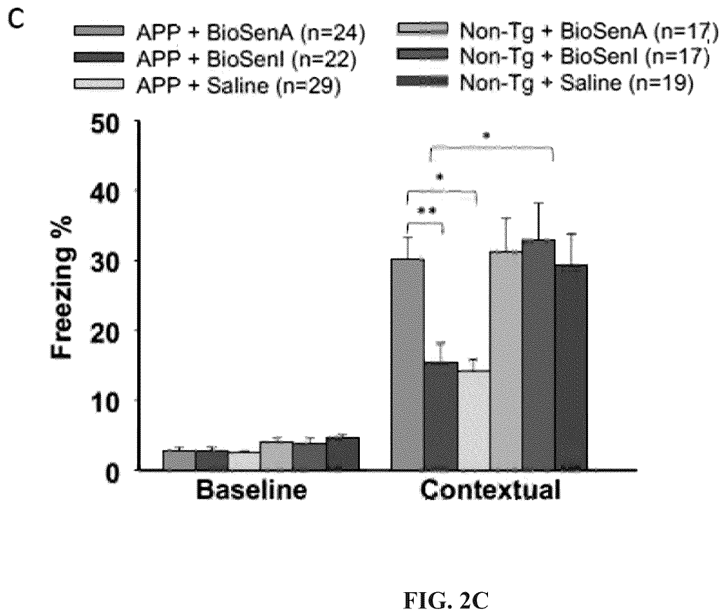

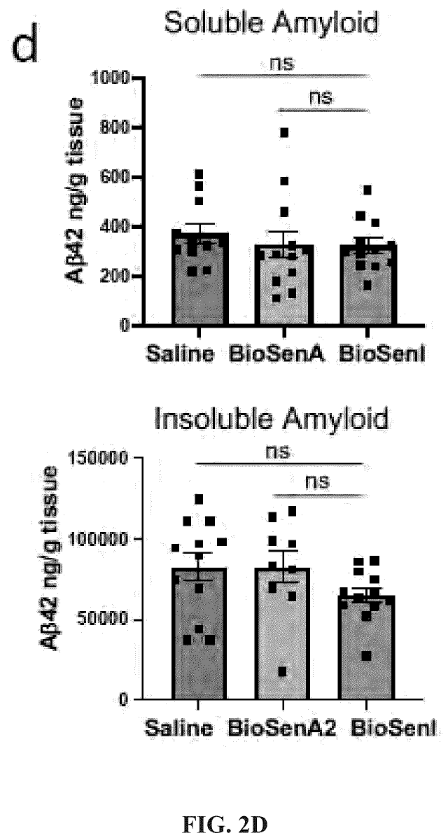

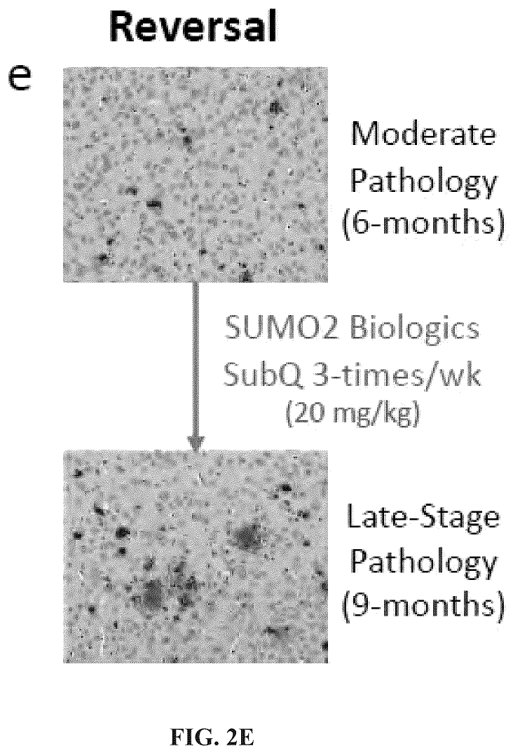

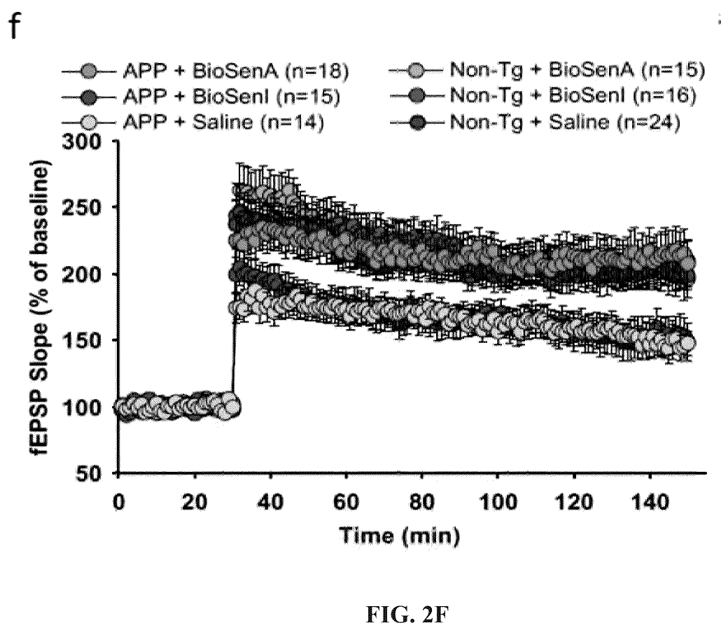

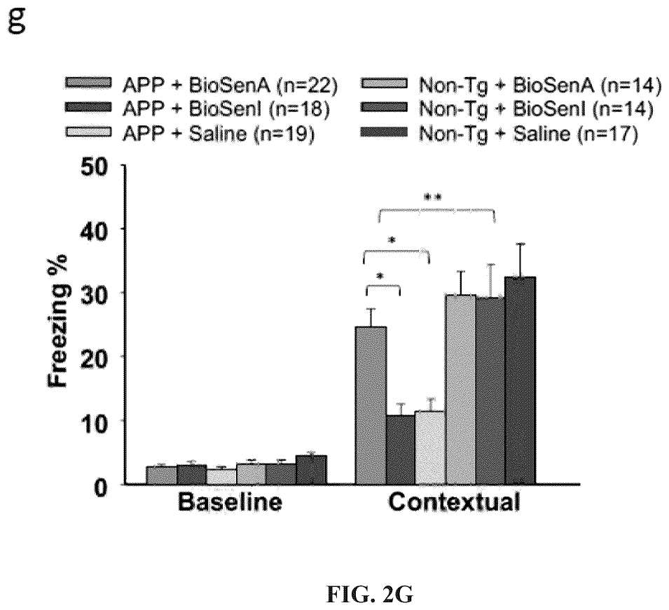

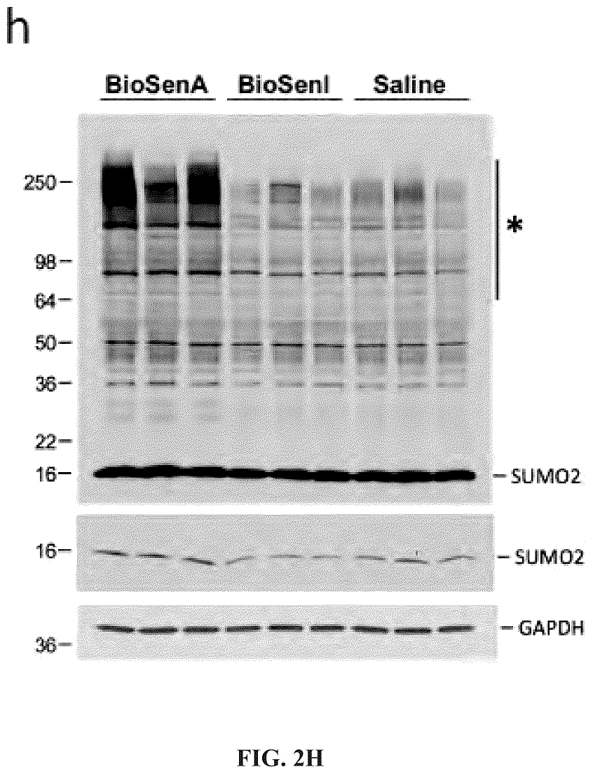

Testing was conducted to examine the ability of BioSenA to intervene in a prophylactic paradigm when amyloid pathology corresponded to the equivalent of early-stage deposition as well as in a reversal paradigm in a late-stage of amyloid deposition. To assess prophylactic efficacy, a cohort of amyloid transgenic mice were treated with the active BioSenA beginning at 3-months of age prior to the onset of amyloid accumulation and behavioral impairments (FIG. 2A). Separate groups of APP transgenics were administered an inactive form of the recombinant SUMO2 analogue, BioSenI, or saline and compared to non-transgenic (Non-Tg) animals receiving comparable treatments. BioSenA and BioSenI (20 mg/kg) were administered 3-times/week for 3-months. Examination of LTP demonstrated that BioSenA prevented the onset of synaptic dysfunction (FIG. 2B). APP transgenics that received inactive BioSenI or saline exhibited a decline in LTP. No changes were observed in Non-Tg mice that underwent a similar treatment regimen. The ability of BioSenA to preserve normal learning and memory were similarly reflected in contextual fear conditioning (FIG. 2C).

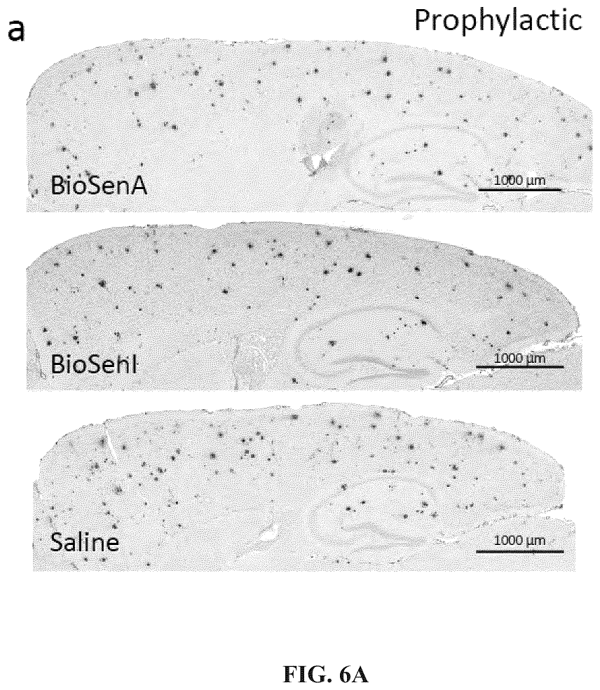

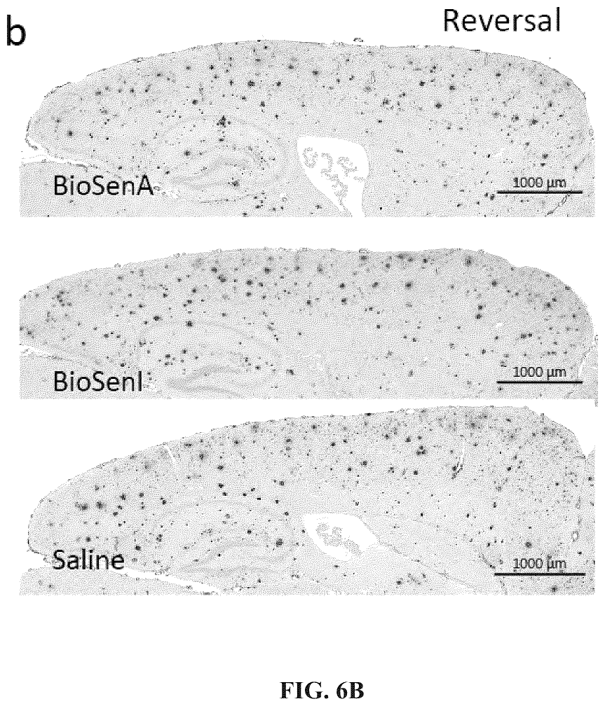

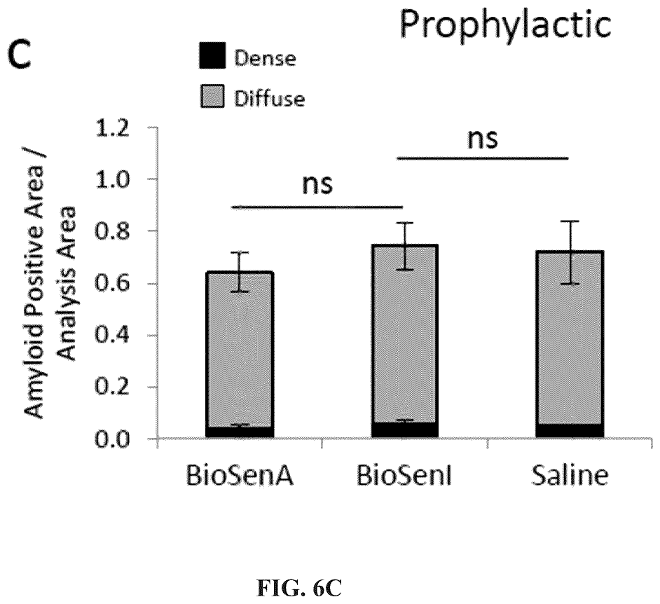

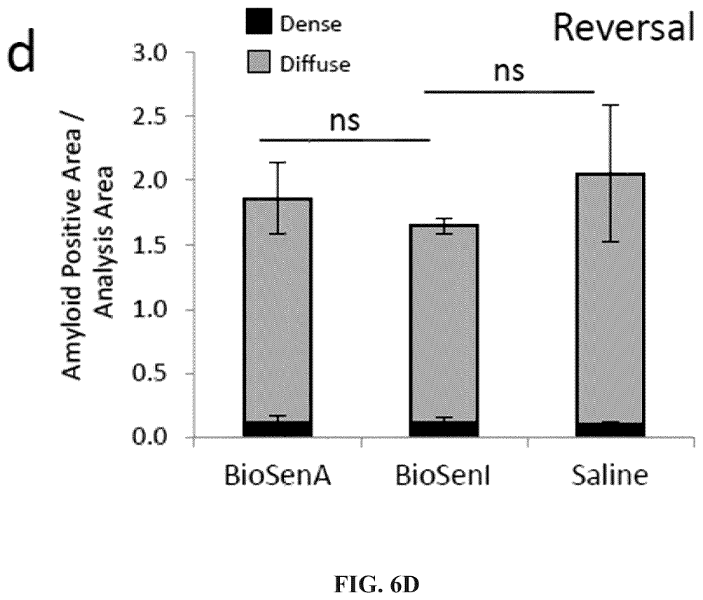

APP transgenic mice receiving BioSenA maintained their cognitive abilities and were not significantly different from the Non-Tg animals. However, the BioSenI or saline treated animals displayed the expected memory defects associated with APP transgenics. As with the SUMO2-APP animals, treatment with the recombinant SUMO2 analogue BioSenAdid not alter the underlying amyloid pathology as indicated by the similar levels of soluble and insoluble Aβ42 (FIG. 2D). Image analysis of amyloid immunohistochemistry further supported this conclusion as the dense and diffuse plaques were unchanged in APP-Tg mice that were treated with BioSenA, BioSenI or Saline (FIGS. 6A and 6C).

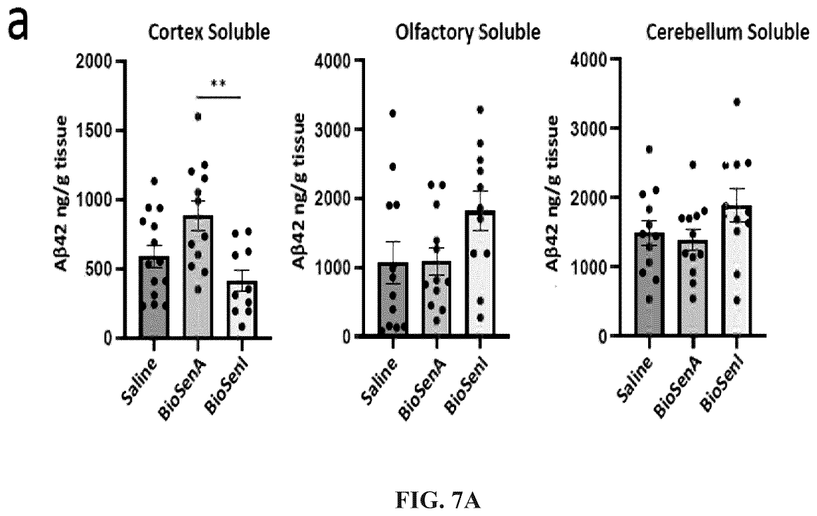

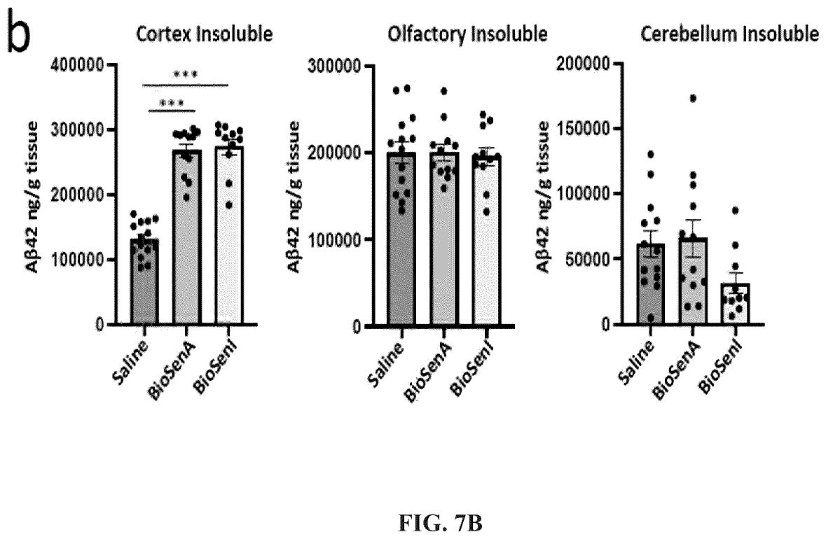

For reversal of late-stage AD, treatments were initiated in APP transgenic mice beginning at 6-months of age when amyloid pathology was established and LTP and cognitive deficits were observed (i.e., endpoint for the prophylactic study; see FIGS. 2B and 2C). BioSenA was administered for 3-months and examined at 9-months of age when the phenotype had advanced to severe AD. Control groups were APP transgenics treated with BioSenI or saline and compared to Non-Tg mice. For APP mice, it was found that BioSenA was able to reverse LTP deficits and restore synaptic function to indistinguishable from Non-Tg animals (FIG. 2F). Comparable effects were observed for cognitive assessments where BioSenA reversed the impairments as determined by fear conditioning at the 9-month endpoint (FIG. 2G). Consistent with the prevention study, image analysis of plaque densities indicated no change in amyloid loads for the BioSenA, BioSenI or Saline treated APP transgenics (FIGS. 6B and 6D). Quantification of the soluble and insoluble Aβ42 levels confirmed the lack of an impact on amyloid loads (FIGS. 7A and 7B). The one exception was the cortical insoluble Aβ where elevated levels were observed for both BioSenA and BioSenI. This may be due to an increase in amyloid aggregates that were not observable by immunocytochemistry.

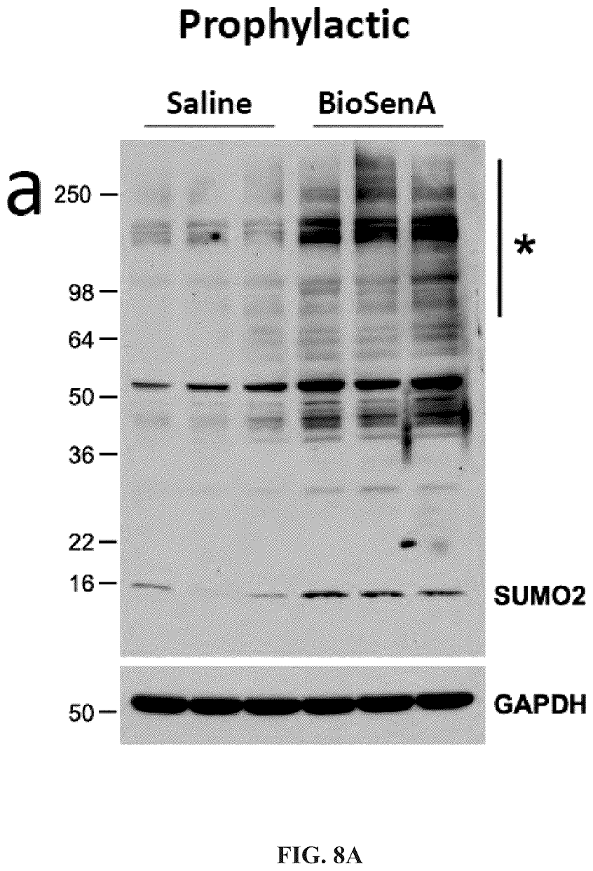

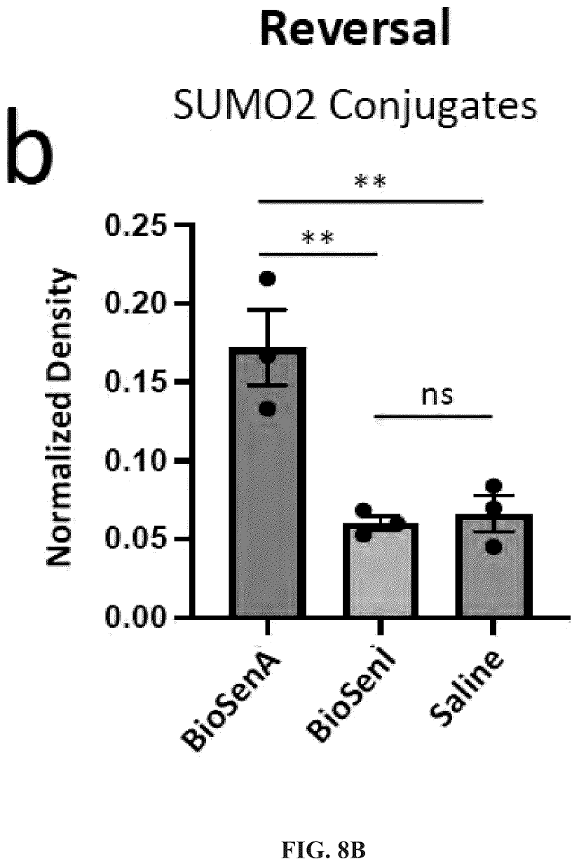

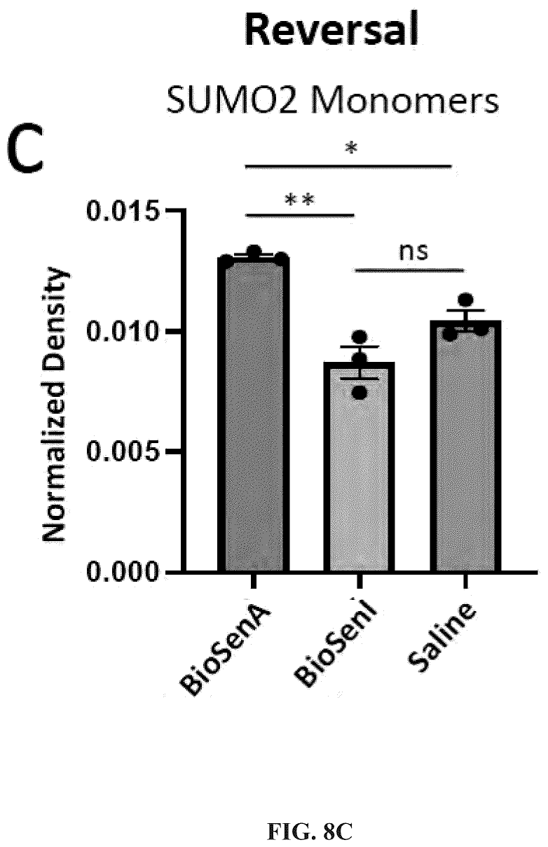

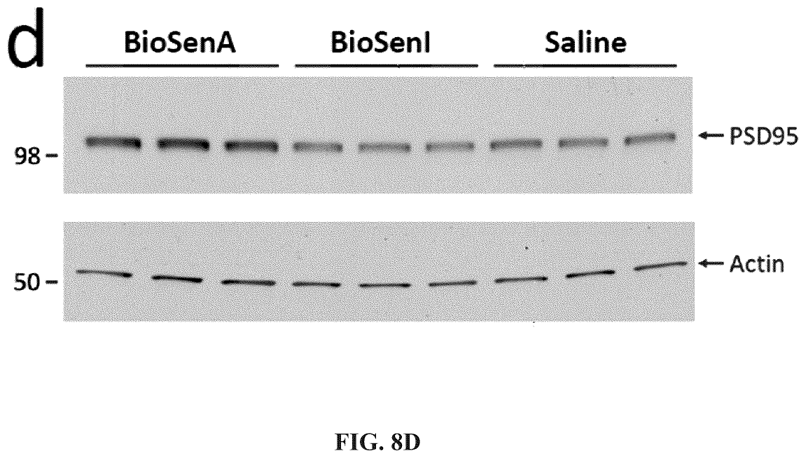

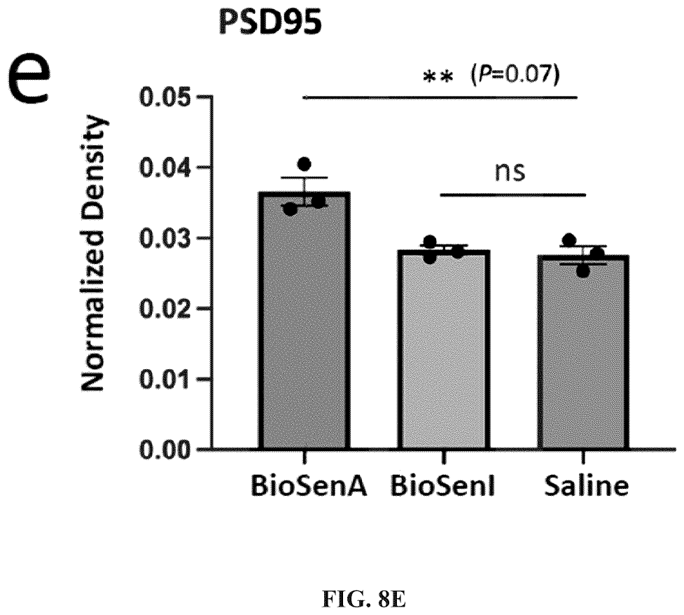

Unlike most amyloid-based therapeutics, the recombinant SUMO2 analogue does not enhance the clearance of Aβ or reduce its production by altering amyloid processing. BioSenA administration for prevention and reversal trials increased the levels of free SUMO2 and enhances conjugation in the brain (FIG. 2H and FIGS. 8A, 8B and 8C). The outcome of the enhanced SUMO2 conjugation appears to be an increase in synaptic density and/or stability. This is illustrated by the elevated levels of the postsynaptic PSD95, in the APP mice from the Reversal study (FIGS. 8D and 8E). Quantification of PSD95 levels from a subset of animals demonstrated an increase in BioSenA treated animals compared to those receiving BioSenI or Saline.

As shown in FIG. 9, the SUMO2 mimetic polypeptide BiosenA (partial SUMO2 sequence) rescues the tau- and amyloid beta-oligomer induced deficit in LTP. Hippocampal slice perfusion with recombinant oligomeric tau 4R/2N (50 nM, 20 min prior to the theta burst) or Amyloid-beta (200 nM, 20 min prior to the theta burst) reduces CA3-CA1 LTP in C57B16 mice. Perfusion with the SUMO2 mimetic polypeptide BiosenA (1 μg/ml,) concurrently with tau or Abta restored LTP to control levels (FIG. 9), suggesting that binding of the mimetic peptide to tau or Amyloid-beta in the extra- and/or intra-cellular space protects synapses from acute toxicity induced by tau and amyloid-beta oligomers. Conversely, BiosenA alone did not affect LTP, suggesting that SUMO2 mimetic is not modifying LTP through alternative mechanisms.

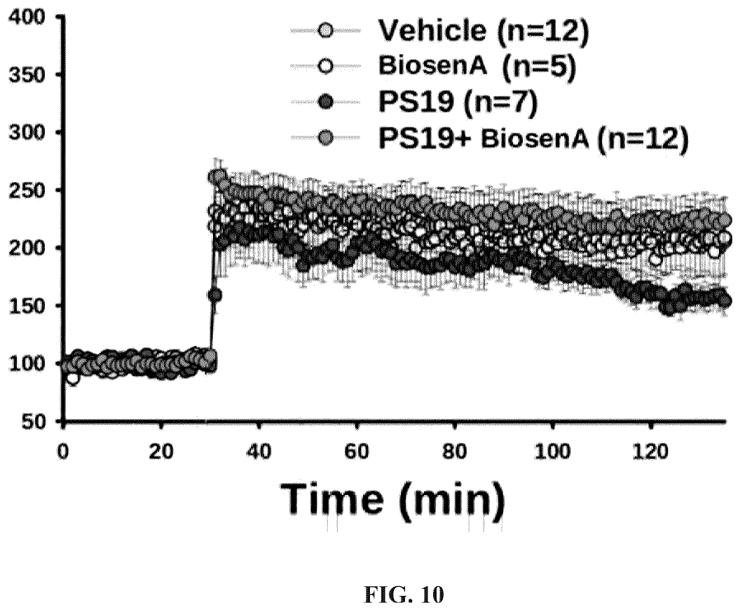

As shown in FIG. 10, the SUMO2 mimetic polypeptide BiosenA (full SUMO2 sequence) rescues the mutant tau-induced deficit in LTP. Chronic treatment of the P301S model of tau deposition (a.k.a. PS19 mouse) with the SUMO2 mimetic polypeptide BiosenA (20 mg/Kg, subcutaneously) from the age of 6 months until the age of 8 months rescues the CA3-CA1 LTP (FIG. 10), suggesting that binding of the mimetic peptide to tau in the extra- and/or intra-cellular space protects synapses from chronic toxicity induced by tau. Conversely, BiosenA alone did not affect LTP in non-transgenic littermates, suggesting that SUMO2 mimetic is not modifying LTP through alternative mechanisms

Cumulatively, the findings of this investigation have shown that BioSenA has high brain bioavailability and an excellent safety profile. Administration of the recombinant SUMO2 analogue results in the prevention and reversal of LTP and cognitive deficits associated with mild and moderate AD. BioSenA treatment has no impact on the extent of amyloid pathology but likely acts by counteracting the toxic effects of Aβ that are responsible for synaptic loss and dysfunction. BioSenA represents an effective therapy to slow the progression of the disease and restore cognitive function in patients that have been diagnosed with later-stage AD. These findings open a new avenue for the treatment of AD and other tauopathies which may be particularly effective either alone or in conjunction with other approaches targeting Ab, such as immunotherapies. Such combinations would provide enhanced synaptic function with an effective amyloid clearance strategy.

Alzheimer's disease (AD) is characterized by neuronal loss, extracellular senile plaques and intracellular neurofibrillary tangles, leading to memory loss. AD purportedly begins as a synaptic disorder produced at least in part, by Aβ (Science 298, 789-791 (2002); herein incorporated by reference in its entirety). Aβ-induced reduction in long-term-potentiation (LTP), a physiological correlate of synaptic plasticity that is thought to underlie learning and memory.

Alzheimer's disease (AD) is a chronic progressive neurodegenerative disorder, in which the earliest stages are thought to be linked to synaptic dysfunction leading to memory disorders. In this regard, β-amyloid (Aβ) has been found to inhibit memory (Proc Natl Acad Sci USA, 2006. 103:8852-7; Nat Neurosci, 2005. 8:79 84; each herein incorporated by reference in its entirety) and its cellular model, long-term potentiation (LTP) (Neuroreport, 1997. 8:3213-7; Eur J Pharmacol, 1999. 382:167-75; Proc Natl Acad Sci USA, 2002. 99:13217-21; Nature, 2002. 416:535-9; J Neurosci Res, 2000. 60:65-72; Proc Natl Acad Sci USA, 1998. 95:6448-53; each herein incorporated by reference in its entirety). Aβ is the proteolytic product of a larger precursor protein, the amyloid precursor protein (APP), which in its mutant form has been found to be implicated in familial AD (FAD) (Nature, 1987. 325:733-6; herein incorporated by reference in its entirety). Subsequently, two AD associated genes, presenilin 1 (PS1) and presenilin 2 (PS2) (Nature, 1995. 375:754-60; Science, 1995. 269:970-3; each herein incorporated by reference in its entirety) were found to be involved in FAD as well (Prog Neurobiol, 2000. 60:363-84; herein incorporated by reference in its entirety). Presenilins are part of the γ-secretase complex responsible for cleaving APP and producing the Aβ42 peptide (Neurosci Lett, 1999. 260:121-4; herein incorporated by reference in its entirety).

AD is characterized neuropathologically by neuronal loss, extracellular senile plaques (SPs) and intracellular neurofibrillary tangles (NFTs). SPs are chiefly comprised of Aβ aggregates. The major component of NFTs is the microtubule binding protein tau. Clinically, AD is characterized by cognitive dysfunction and begins as a synaptic disorder that involves progressively larger areas of the brain over time (Histol Histopathol, 1995. 10(2): p. 509-19; herein incorporated by reference in its entirety). An emerging view of the processes involved in synaptic impairment shows that the subtlety and variability of the earliest amnesic symptoms, occurring in the absence of any other clinical signs of brain injury, can be due to discrete changes in the function of a single synapse, produced at least in part, by Aβ (Neuroreport, 1997. 8(15): p. 3213-7; Eur J Pharmacol, 1999. 382(3): p. 167-75; Proc Natl Acad Sci USA, 2002. 99(20): p. 13217-21; Nature, 2002. 416(6880): p. 535-9; herein incorporated by reference in its entirety).

A target for developing a causal therapy for Alzheimer's disease is represented by synapses. Synaptic alterations are highly correlated with the severity of clinical dementia (Histol Histopathol, 1995. 10(2): p. 509-19; Science, 2002. 298(5594): p. 789-91; each herein incorporated by reference in its entirety), whereas other important variables such as senile plaques and neurofibrillary tangles are involved to a lesser extent (Histol Histopathol, 1995. 10(2): p. 509-19; herein incorporated by reference in its entirety). The importance of synaptic alterations in AD has been confirmed by studies of transgenic (Tg) mouse models of AD (Neurochem Res, 2003. 28(7): p. 1009-15; herein incorporated by reference in its entirety), as well as of long-term potentiation (LTP), a widely studied cellular model of learning and memory (L&M) (Nature, 2002. 416(6880): p. 535-9; herein incorporated by reference in its entirety), which is impaired following application of amyloid-β (Aβ) both in slices and in vivo (Neurochem Res, 2003. 28(7): p. 1009-15; Neuroreport, 1997. 8(15): p. 3213-7; J Neurophysiol, 2001. 85(2): p. 708-13; Eur J Pharmacol, 1999. 382(3): p. 167-75; J Neurosci, 2001. 21(4): p. 1327-33; J Neurosci, 2001. 21(15): p. 5703-14; Proc Natl Acad Sci USA, 2002. 99(20): p. 13217-21; Nature, 2002. 416(6880): p. 535-9; J Neurosci, 2005. 25(29): p. 6887-97; each herein incorporated by reference in its entirety). Aβ has been found to markedly inhibit LTP. Electrophysiological studies using Tg, human Aβ producing mice have often revealed significant deficits in basal synaptic transmission and/or LTP in the hippocampus (Ann Neurol, 2004. 55(6): p. 801-14; Nat Neurosci, 1999. 2(3): p. 271-6; J Neurosci, 2001. 21(13): p. 4691-8; Proc Natl Acad Sci USA, 1999. 96(6): p. 3228-33; Neurobiol Dis, 2002. 11(3): p. 394-409; Brain Res, 1999. 840(1-2): p. 23-35; J Biol Chem, 1999. 274(10): p. 6483-92; Nature, 1997. 387(6632): p. 500-5; each herein incorporated by reference in its entirety).

Tauopathies can be divided into three groups, on the basis of the isoforms that constitute the abnormal filaments. Known Tau isoform compositions of NFTs:

### 3R+4R

Alzheimer's disease (AD), Amyotrophic lateral sclerosis/parkinsonism-dementia complex (ALS), Anti-lgLON5-related Tauopathy, Chronic traumatic encephalopathy (CTE), Diffuse neurofibrillary tangles with calcification, Down's syndrome, Familial British dementia (FBD), Familial Danish dementia (FDD), Gerstmann-Straussler-Scheinker disease (GSS), Niemann-Pick disease, type C (NPC), Nodding syndrome, Non-Guamanian motor neuron disease with neurofibrillary tangles, Postencephalitic parkinsonism, Primary age-related tauopathy (PART), Progressive ataxia and palatal tremor, SLC9A6-related parkinsonism, Tangle-only dementia (TD), Familial frontotemporal dementia and parkinsonism (FTDP) with mutations V337M and R406W.

### 4R

Age-related Tau astrogliopathy (ARTAG), Argyrophilic grain disease (AG), Corticobasal degeneration (CBD), Guadeluopean parkinsonism, Globular glial Tauopathy (GGT), Hippocampal Tauopathy, Huntington's disease, Progressive supranuclear palsy (PSP), Trauma related Tau astrogliopathy, Familial frontotemporal dementia and parkinsonism (mutations P301S, intronic mutations, coding region mutations in exon 10).

### 3R

Pick's disease (PiD), Familial frontotemporal dementia and parkinsonism (mutations G272V and Q336R).

Galina Limorenko, Hilal A Lashuel, To target Tau pathologies, we must embrace and reconstruct their complexities, Neurobiology of Disease, Volume 161, 2021, 105536, ISSN 0969-9961, https://doi.org/10.1016/).nbd.2021.105536.

Other relevant applications related to AD and pharmacological studies are described, for example, in the following publications, each of which is incorporated herein by reference in its entirety:
Alam J J, Compositions and method for treating Alzheimer's Disease, WO 2012/154814;
Alamed J. et al., (2006) Two-day radial-arm water maze learning and memory task; robust resolution of amyloid-related memory deficits in transgenic mice. Nat Protoc 1: 1671-1679;
Masliah E (1995) Mechanisms of synaptic dysfunction in Alzheimer's disease. Histol Histopathol 10: 509-519;
Oddo S et al., (2003) Triple-transgenic model of Alzheimer's disease with plaques and tangles: intracellular Abeta and synaptic dysfunction. Neuron 39: 409-421;
Rowan M J et al., (2003) Synaptic plasticity in animal models of early Alzheimer's disease. Philos Trans R Soc Lond B Biol Sci 358: 821-828;
Selkoe D J (2002) Alzheimer's disease is a synaptic failure. Science 298: 789-791;
Selkoe D J (2008) Soluble oligomers of the amyloid beta-protein impair synaptic plasticity and behavior. Behav Brain Res 192: 106-113; and
Teich A F and Arancio O (2012) Is the Amyloid Hypothesis of Alzheimer's Disease Therapeutically Relevant? Biochem J. 446: 165-177.

### Pharmaceutical Compositions, Methods of Administration and Combination Treatments

In some embodiments, the recombinant SUMO2 analogue can be supplied in the form of a pharmaceutical composition, comprising an isotonic excipient prepared under sufficiently sterile conditions for human administration. Choice of the excipient and any accompanying elements of the composition will be adapted in accordance with the route and device used for administration. In some embodiments, a composition comprising a recombinant SUMO2 analogue can also comprise, or be accompanied with, one or more other ingredients that facilitate the delivery or functional mobilization of the treatment.

These methods described herein are by no means all-inclusive, and further methods to suit the specific application is understood by the ordinary skilled artisan. Moreover, the effective amount of the compositions can be further approximated through analogy to compounds known to exert the desired effect.

According to the present disclosure, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

Pharmaceutical compositions for use in accordance with the invention can be formulated in conventional manner using one or more physiologically acceptable carriers or excipients. The therapeutic compositions of the invention can be formulated for a variety of routes of administration, including systemic and topical or localized administration. Techniques and formulations generally can be found in Remmington's Pharmaceutical Sciences, Meade Publishing Co., Easton, Pa (20th ed., 2000), the entire disclosure of which is herein incorporated by reference. For systemic administration, an injection is useful, including intramuscular, intravenous, intraperitoneal, and subcutaneous. For injection, the therapeutic compositions of the invention can be formulated in liquid solutions, for example in physiologically compatible buffers, such as PBS, Hank's solution, or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

Any of the therapeutic applications described herein can be applied to any subject in need of such therapy, including, for example, a mammal such as a dog, a cat, a cow, a horse, a rabbit, a monkey, a pig, a sheep, a goat, or a human. In some embodiments, the subject is a human.

A pharmaceutical composition of the present disclosure is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

The dosage administered can be a therapeutically effective amount of the composition sufficient to result in treatment of a AD, prevention of AD, or a decrease in symptoms associated with AD, and can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

Toxicity and therapeutic efficacy of therapeutic compositions of the present invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapeutic agents that exhibit large therapeutic indices are useful. Therapeutic compositions that exhibit some toxic side effects can be used.

Experimental animals can be used as models for human disease. For example, mice can be used as a mammalian model system. The physiological systems that mammals possess can be found in mice, and in humans, for example. Certain diseases can be induced in mice by manipulating their environment, genome, or a combination of both.

Administration of a recombinant SUMO2 analogue is not restricted to a single route but may encompass administration by multiple routes. Multiple administrations may be sequential or concurrent. Other modes of application by multiple routes will be apparent to one of skill in the art.

### Experimental Results

FIG. 1 shows that elevated SUMO2 protects against amyloid-induced cognitive decline and defects in synaptic plasticity in vivo. FIG. 1A provides immunoblot analysis of transgenic mice expressing human SUMO2 by the prion cos-tet promoter within different brain regions (Ob—olfactory bulb; Cx—cortex; Cb—cerebellum; Hip—hippocampus) showing increased levels of free SUMO2 and no significant increases in high molecular weight conjugates. Control untransfected (Ct) and HA-tagged SUMO2 transfected (Tf) HEK293 cells were used as positive controls. FIG. 1B shows that SUMO2 overexpression protected against LTP impairment of APP mice (2-way ANOVA: Non-Tg vs. APP F(1,17)=9.960, P=0.0058; APP vs. SUMO2 APP F(1,17)=5.713, P=0.0287; Non-Tg vs. SUMO2 F(1,16)=0.9424, P=0.3461). “n” indicated the number of slices in this and the following graphs. FIG. 1C shows that contextual fear conditioning as a measure of associative memory impairments in APP mice were prevented by SUMO2 overexpression. One-way ANOVA with Tukey's multiple comparisons test, ** p<0.01; * p<0.05. Data are means±s.e.m. FIG. 1D provides quantification of soluble and insoluble Aβ42 as determined by sandwich ELISA, which indicated no significant difference in amyloid loads in the cerebellum (Cb) and cortex-hippocampus (C/H) extracts; ns=not significant, Dunnett's multiple comparison test after one-way ANOVA. As shown in FIG. 1E, immunoblotting for SUMO2 demonstrated an increase in SUMO2 higher molecular weight conjugated proteins (*) in the SUMO2-APP transgenics and elevated free SUMO2 in synaptic compartments in whole brain homogenates for SUMO2-APP transgenics as compared to APP mice only. Elevated SUMO2 was also observed in isolated synaptosomes suggesting a targeting to synapses and potential impact on activity. All procedures were reviewed and approved by the University of Toronto and Columbia University Animal Care and Use Committee.

FIG. 2 illustrates that the recombinant analogue, BioSenA, mimics SUMO2 activity with respect to preventing and reversing impairments in synaptic plasticity and cognition. FIG. 2A shows a schematic of the prophylactic therapy time course for treatments with recombinant SUMO2 analogues, BioSenA and BioSenI (20 mg/kg; SC. 3×/wk) and saline controls. FIG. 2B shows that APP mice exhibit LTP deficits in comparison to littermates Non-Tg mice (F(1,21)=5.771; P=0.0256). BioSenA prevented the LTP impairment in APP mice as compared to treatment with inactive BioSenA (F(1,21)=7.636; P=0.012) or saline (F(1,19)=6.119; P=0.023) and exhibited potentiation similar to Non-Tg mice receiving comparable treatments (F(1,21)=0.007; P=0.938). As shown in FIG. 2C, deficit in associative memory as determined by contextual fear conditioning was prevented by BioSenA administration as compared to BioSenI or saline treated APP animals (1-way ANOVA followed by Bonferroni's multiple comparisons: F(5,120)=5.940, P<0.0001; APP+Saline vs Non-Tg+Saline: P=0.0353; APP+BioSenA vs APP+BiosenI: P=0.012; APP+BioSenA vs. APP+Saline: P=0.002). No difference was found in the baseline freezing between the different groups (1-way ANOVA for all groups: F(5,120)=2.186, P=0.06). FIG. 2D provides ELISA quantification of soluble and insoluble Aβ42 in cortex-hippocampal homogenates, which indicated no changes in amyloid loads as shown by the insoluble and soluble Aβ42 levels; n.s.—not significant, Tukey's multiple comparisons after one-way ANOVA. FIG. 2E provides timelines for the Reversal study with BioSenA/BioSenI and treatment of APP transgenics with pre-existing amyloid pathology and LTP/cognitive impairments at 6-months (moderate AD) until 9-months of age (severe AD). FIG. 2F shows that LTP impairment in APP mice (APP+Saline vs Non-Tg+Saline: F(1,36)=9.241, P=0.004) was reverted by BioSenA treatment as compared to animals receiving inactive BioSenI (F(1,31)=7.077; P=0.0123) or Saline (F(1,30)=8.005; P=0.0082); and exhibited potentiation similar to Non-Tg mice receiving comparable treatments (F(1,31)=0.071; P=0.792). FIG. 2G shows that associative memory deficits in APP mice (APP+Saline vs Non-Tg+Saline: P=0.002), assessed by contextual fear conditioning were reversed by BioSenA relative to transgenics treated with BioSenI (P=0.037) or saline (P=0.02) (1-way ANOVA for all groups: F(5,98)=7.183, P=0<0.0001. No difference was found in baseline activity (1-way ANOVA for all groups: F(5,98)=1.719, P=0.137). As shown in FIG. 2H, SUMO2 immunoblotting revealed an increase in high molecular weight conjugates (*) for whole brain homogenates from BioSenA-treated APP transgenics relative to BioSenI or Saline treated mice and free SUMO2 was modestly increased in mice administered BioSenA (quantification and statistical significance in provided in FIG. 7).

As shown in FIG. 3A, SUMO2 homozygous (SUMO2+/+) and heterozygous (SUMO2+/−) transgenic mice displayed normal LTP compared to non-transgenic (Non-Tg) animals (Two-way ANOVA: non-Tg vs. SUMO2+/+F(1,13)=2.719, P=0.1231; non-Tg vs. SUMO2+/−F(1,16)=1.022, P=0.3270; SUMO2+/+vs. SUMO2+/−F(1,17)=1.114, P=0.3060). “n” indicates the number of hippocampal slices examined. FIG. 3B shows that contextual fear conditioning indicated no significant differences (ns) between homozygous or heterozygous SUMO2 transgenics and Non-Tg animals. FIG. 3C shows that sensory thresholds assessment did not reveal any significant difference between groups of test mice (1st visible response F(3,59)=0.1975 P=0.8977; 1st motor response F(3,59)=0.6767 P=0.5698; 1st vocal response F(3,59)=0.2042 P=0.8931).

FIG. 4A provides representative images showing amyloid immunohistochemistry in the APP and SUMO2-APP transgenics displaying comparable plaque densities (scale bars, 500 μm). FIG. 4B provides quantitative image analysis for dense and diffuse amyloid plaques, which revealed comparable levels in APP and SUMO2-APP transgenic mice indicating SUMO2 overexpression had no effect on the amounts of dense plaques (black) and diffuse (gray) amyloid deposits were modestly increased in the SUMO2-APP mice (* p<0.05; Tukey's multiple comparisons test after one-way ANOVA). FIG. 4C shows that amyloid processing in APP and SUMO2-APP transgenics was unchanged based upon APP full-length (APP-FL) and C-terminal fragments (APP-CTF) and no differences in the E2 ligase, Ubc9, were observed. FIG. 4D shows that ELISA assays for the secreted APP b-fragment (sAPPbeta-sw) at 6- and 9-months exhibited no statistical differences for APP and SUMO2-APP transgenics. FIG. 4E shows that endogenous murine Aβ42 remained unchanged in SUMO2 over-expressing mice relative to Non-Tg animals (6-months of age), Dunnett's multiple comparison test after one-way ANOVA.

As shown in FIG. 5, BioSenA (full SUMO2 sequence) levels were examined following intravenous (IV) or subcutaneous (SQ) administration. Wild-type C57BL/6 mice were administered a single injection of C-terminally His-tagged BioSenA (20 mg/Kg) and samples were analyzed at various time points. FIG. 5A: Quantification of the plasma using His-ELISA assays showed a rapid elevation in BioSenA following IV injection that peaked at approximately 30 mins and was completely cleared from the periphery after 4 hrs. FIG. 5B: Subcutaneously (SQ) injected BioSenA peaked at 1 hr and gradually decreased with detectable levels of ˜15 ng/ml at 4 hrs post-injection. Peak plasma levels were higher in the IV-injected group at ˜115 ng/ml as compared to SC injected mice at ˜40 ng/ml plasma indicating that IV administered BioSenA results in higher plasma concentrations, but these are eliminated more rapidly than in mice receiving SQ injected biologic. FIG. 5C: BioSenA concentrations in the cortex following IV administration gradually increased and reached a maximum of ˜60 ng/g tissue 24 hrs post-injection. FIG. 5D: SQ injected BioSenA rapidly increased in the cortex and reached a maximal level of ˜125 ng/g tissue at the 4 hr time point and significant levels were observed 24 hrs post-injection. N=3 mice per time point and data are means±s.e.m.

Representative immunocytochemistry images for the Prophylactic (FIG. 6A) and Reversal (FIG. 6B) treatments show similar plaque densities in APP mice treated with BioSenA, BioSenI or Saline. Sections were stained for Aβ indicating the comparable amyloid plaque loads in the cortex and hippocampus. Amyloid plaque loads were assessed by immunohistochemistry and image analysis of the cortex and hippocampus. Dense plaque cores (black) and diffuse halos (gray) for APP-Tg mice treated prophylactically at the (FIG. 6C) pre-pathology and cognitively normal stages (3-months of age; n=3; 5 sections/animal) or (FIG. 6D) the reversal treatment after amyloid pathology and cognitive impairments were well-established (6-months of age; n=3; 5 sections/animal) exhibited comparable amyloid loads following 3-months administration of the active BioSenA, inactive BioSenI or Saline (Tukey's multiple comparisons test after one-way ANOVA, ns; nonspecific). Scale bars, 1000 μm.

Amyloid Precursor Protein processing was largely unchanged as shown by (FIG. 7A) Soluble Aβ42 levels in homogenates from dissected cortex, olfactory bulb and cerebellum as determined by sandwich ELISA, although a modest decrease was observed in the cortices of APP-Tg mice treated with BioSenI. (FIG. 7B) Insoluble Aβ42 was elevated in APP-Tg in the cortex for mice receiving BioSenA and BioSenI suggesting increased aggregation over the long term treatment of the reversal preclinical testing. Insoluble Aβ42 as an indicator of amyloid loads was unchanged in the olfactory bulb and cerebellum. **;p<0.01, ***;p<0.001 by Tukey's multiple comparisons test after one-way ANOVA.

FIG. 8A: Immunoblotting of whole brain homogenates from the prophylactic treated with BioSenA showing the increase in both high molecular weight SUMO2 conjugated proteins and free SUMO2, possibly corresponding to BioSenA, as compared to saline treated APP animals. Quantification of SUMO2 conjugates (FIG. 8B) and monomeric free SUMO2 (FIG. 8C) in brain homogenates from the reversal study demonstrating the significant increase in SUMOylation in BioSenA treated animals and modest increases in the free SUMO2; ANOVA with post-hoc analyses: **p<0.005; *p<0.01; Tukey's multiple comparison test after one-way ANOVA. FIG. 8D: Immunoblot for PSD95 from brain homogenates (cortex-hippocampus combined) indicating an increase in BioSenA treated mice as compared to BioSenI or Saline suggesting the restoration of synaptic activity. FIG. 8E: Quantification of PSD95 immunoreactivity and the increased levels in BioSenA treated mice; Dunnett's multiple comparison test after one-way ANOVA.

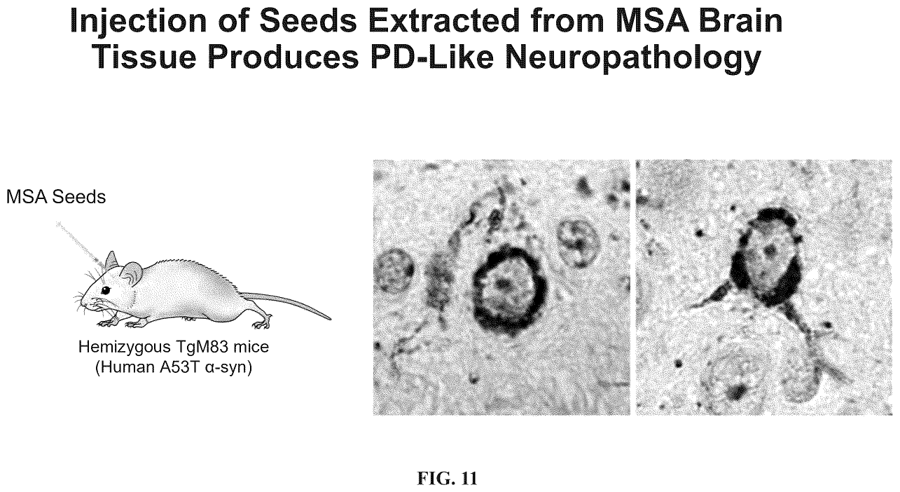

BioSenA Effects in a Model of PD Pathology. To test the effects of BioSenA in a PD model, a single, intracerebral inoculation was used with Multiple System Atrophy (MSA) brain homogenate to hemizygous transgenic M83′ mice expressing human A53T a-synuclein (aSyn). This induces progressive synucleinopathy in 100-150 days, exhibiting bilateral aSyn neuropathology composed of detergent-insoluble, protease-resistant pS129 aSyn in the midbrain, cortex, and brainstem, with progressive quantifiable synaptic deficits and motor abnormalities. FIG. 11 provides photographs of immunohistochemical staining for the phosphorylation at Serine-129 (pS129; brown) showing Parkinson's disease related α-synuclein pathology within neuronals. This rapid, progressive, and reproducible clinicopathological syndrome of MSA-inoculated M83^{+/-} mice is a well-established model of spreading synucleinopathy and an ideal platform to assess therapeutic benefits of the BioSenA biologic. Importantly, widespread pS129 and p62 positive Lewy-like pathology was observed in midbrain, hippocampus, amygdala, cortex, and brainstem. Because the neuropathology is bilateral, this allows the ipsilateral hemisphere to be used for immunofluorescence and the contralateral hemisphere for biochemistry. Moreover, the appearance of motor deficits is apparent in these tests at ˜90 days post-inoculation (dpi), with mortality ensuing at 120-150 dpi. See, generally Menon, S., et al. Viral alpha-synuclein knockdown prevents spreading synucleinopathy. Brain Commun 3, fcab247 (2021) and Lau, A., et al. alpha-Synuclein strains target distinct brain regions and cell types. Nat Neurosci 23, 21-31 (2020).

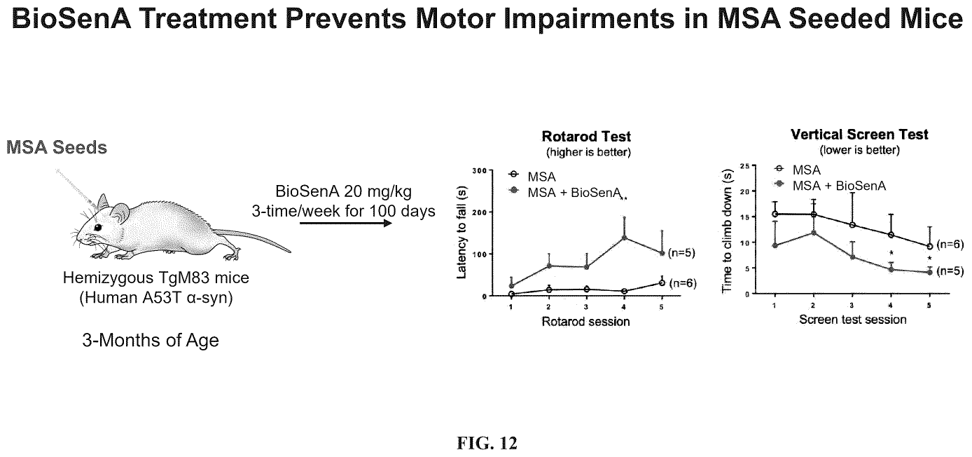

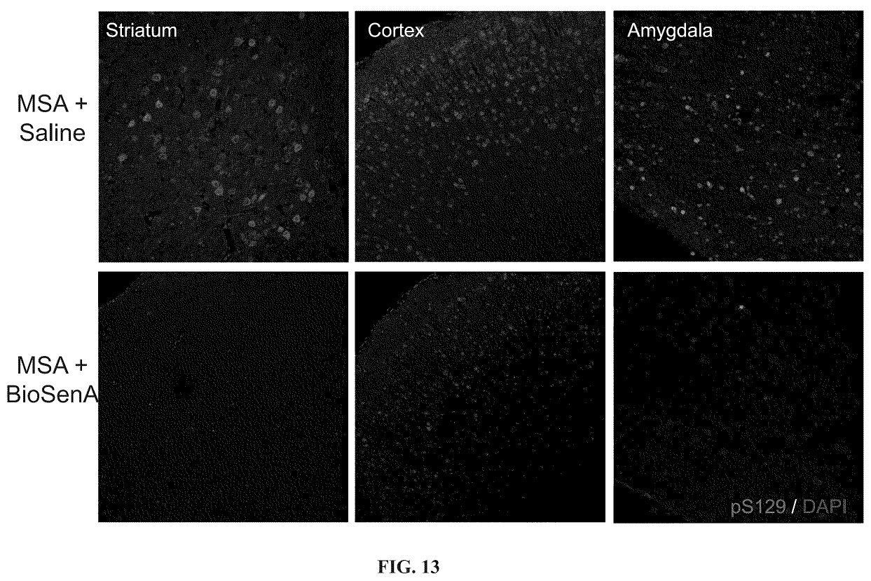

M83^{+/-} transgenics (aged 3-months) were administered 20 mg/kg BioSenA by subcutaneous injections 3-times/week for a period of 100 days. Another group of MSA injected M83 mice were treated with saline over the same time frame and used as controls. The data shows improvements in rotarod and vertical screen test at the 100 days post-injection in animals receiving both MSA-inoculation and BioSenA, compared to the significantly diminished abilities in MSA-inoculated mice that received saline injections (FIG. 12). The rotarod and screen test are routinely used to assess motor function in PD mouse models. Analysis by immunofluorescence indicated that the M83^{+/-} mice that received BioSenA displayed a significant reduction in the levels of phosphorylated aSyn at Serine-129 (pS129; green) which is a clinical marker of PD-related pathology (FIG. 13). M83 transgenics inoculated with MSA extracted seeds that were administered saline exhibited the widespread pathology in various regions of the brain. In contrast, MS83 treated with BioSenA has a significant reduction indicated the ability of BioSenA to prevent the progression of Parkinson's disease related pathology. DAPI (blue) was used as a counterstain. Cumulatively, these findings demonstrate the ability of BioSenA to improve the in vivo PD motor phenotype by directly attenuating the pathology in this mouse model.

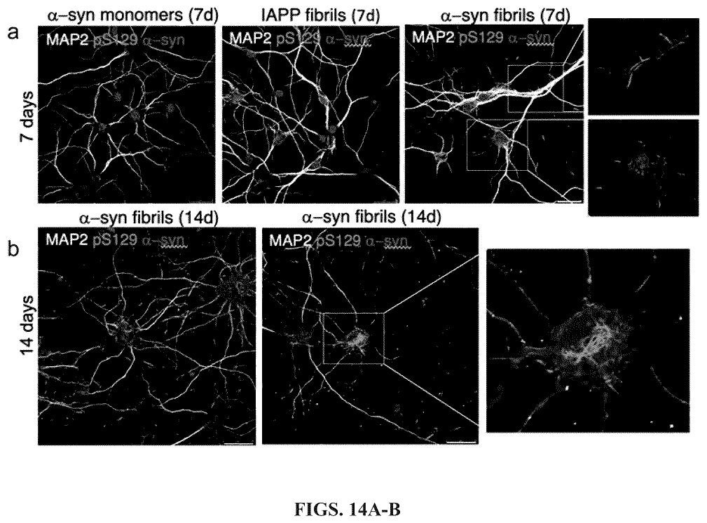

BIOSENA In Vitro Activity: Although no cell culture model is a perfect fit for all three synucleinopathies, it is important to note that hippocampal neurons are affected at later stages of PD, i.e., Stage 4 and in cases with PD dementia, in severe cases of MSA, and more broadly in DLB. These dissociated hippocampal neurons are prepared from wild-type CD1 mice at embryonic day 16-17 and matured for 7 days. Cultures are exposed to 5 μg/ml sonicated aSyn PFF for 24 hrs. These (PFFs) seeded primary neurons display a progressive synucleinopathy initially visible in axons and progressing to the soma after 14 days. FIGS. 14A and 14B show that accumulation of pS129 asyn immunoreactivity (green) progressively increased after 7-days (FIG. 14A) or 14-days FIG. 14B). Exogenous asyn AF555 labeled PFF (red) are still detectable after 14 days in culture. Induction of pathology is dependent on intracellular aSyn because treatment of hippocampal neurons generated from aSyn-deficient mice do not acquire pS129 aSyn pathology. Finally, these hippocampal neurons can be maintained in culture after drug treatment for 2 weeks, having developed widespread axonal and dendritic arborization, and abundant synucleinopathy.

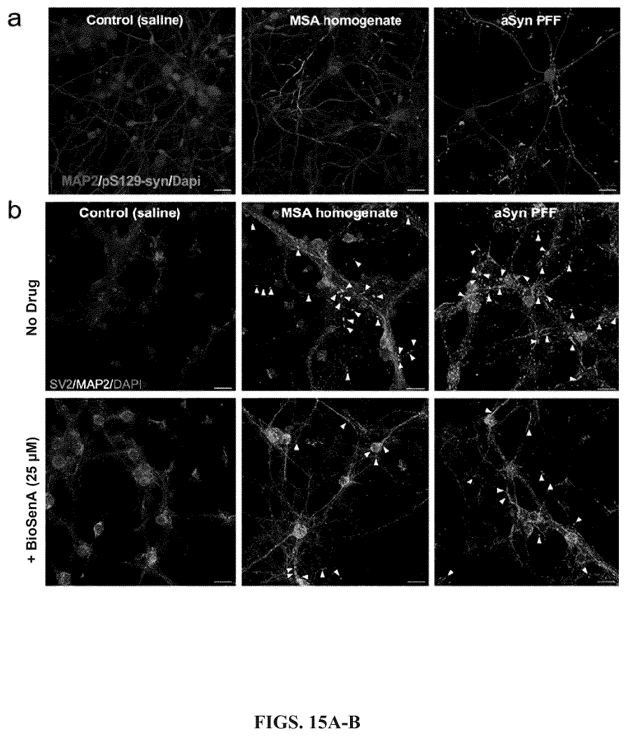

This cell-based model was used to examine the effects of BioSenA on changes in synaptic markers, such as the pre-synaptic SV2A. FIG. 15 shows that BioSenA treatment reduces asyn aggregate-induced accumulation and mislocalization of synaptic protein SV2A. Primary hippocampal neurons were treated for 24 hrs with PBS, MSA homogenate, or asyn preformed fibrils (PFF). After 14 days in culture, (a) neurons accumulated pSer129 asyn pathology, and (b) synaptic vesicle marker SV2 is mislocalized to large puncta and ribbons along axons suggesting disruption of synaptic structure. In the presence of 25 μM BioSenA, the asyn aggregates induced fewer and smaller SV2 inclusions. Following treatment with 25 μm BioSenA for 14 days, the SV2A puncta and ribbon morphology is both smaller and less frequent, ostensibly indicating less synaptic dysfunction (FIGS. 15A and 15B). These findings suggest that BioSenA treatment prevents the downstream effects of aSyn pathology on the impaired trafficking of proteins to the synaptic compartment. In addition, the ability of BioSenA to prevent the spreading of aSyn pathology was assessed using an established propagation assay (see Uchihara, T et al. Propagation of alpha-synuclein pathology: hypotheses, discoveries, and yet unresolved questions from experimental and human brain studies. Acta Neuropathologica 131, 49-73 (2016).

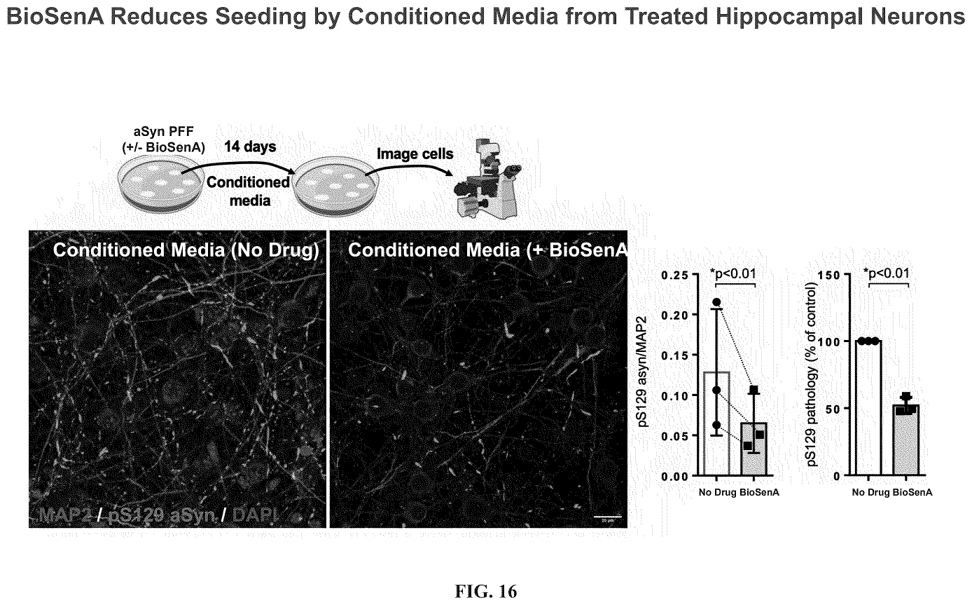

FIG. 16 shows that BioSenA reduces seeding by conditioned media from treated hippocampal neurons. Mouse primary hippocampal neurons were used to examine the mechanism of action for BioSenA and the observed reduction in α-synuclein pathology. Preformed fibrils (PFFs) generated from recombinant human α-synuclein were added to the culture medium to induce intraneuronal aggregation of endogenous α-synuclein over the course of 14-days. Extensive pathology was observed at this timepoint as shown by immunofluorescence staining for the phosphorylated Ser-129 residue (pS129; green). Comparable neuronal cultures exposed to PFFs were treated with BioSenA (25 μM final concentration) that was added to the medium at 48-hour intervals. The conditioned medium from both sets of cells released from the neurons was added to naïve primary neurons to assess the relative levels of α-synuclein seeds in the conditioned medium. Following incubation for 14-days without further treatment, it was observed that cells treated with BioSenA displayed much lower seeding capacity compared to untreated neurons. These data suggest that BioSenA inhibits the release of α-synuclein seeds by cells to prevent the propagation of PD-related pathology. DAPI (blue) was used as a counterstain. The BioSenA treated medium exhibited a significantly reduced ability to seed comparable naïve neurons (FIG. 16). These data are consistent with the ability of BioSenA treatment to reduce the release of aSyn aggregates/seeds and may point towards a potential mechanism of action for the reduction of PD-related pathology in the MSA mouse model.

Spreading of PD Pathology is Attenuated by SUMO2. A commonly used approach to replicate PD pathology is where aSyn preformed fibrils (PFF) are injected into the dorsal striatum to cause retrograde spread of aSyn pathology into the substantia nigra. This model has the advantage of targeting the dopaminergic neurons in the nigra, though resulting motor deficits can be relatively mild and with variable latency 90-180 days. Polinski, N.K. A Summary of Phenotypes Observed in the In Vivo Rodent Alpha-Synuclein Preformed Fibril Model. J Parkinsons Dis (2021).

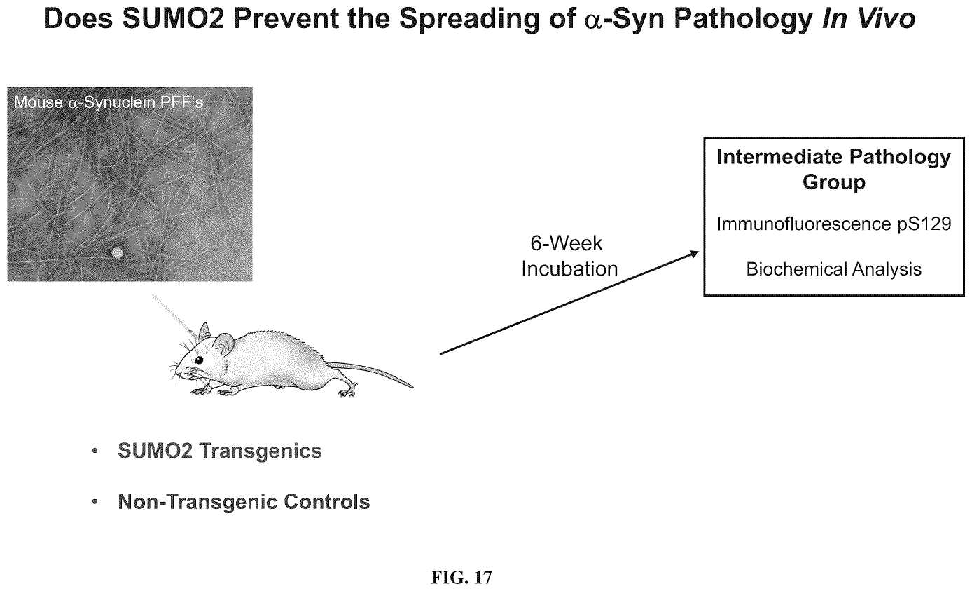

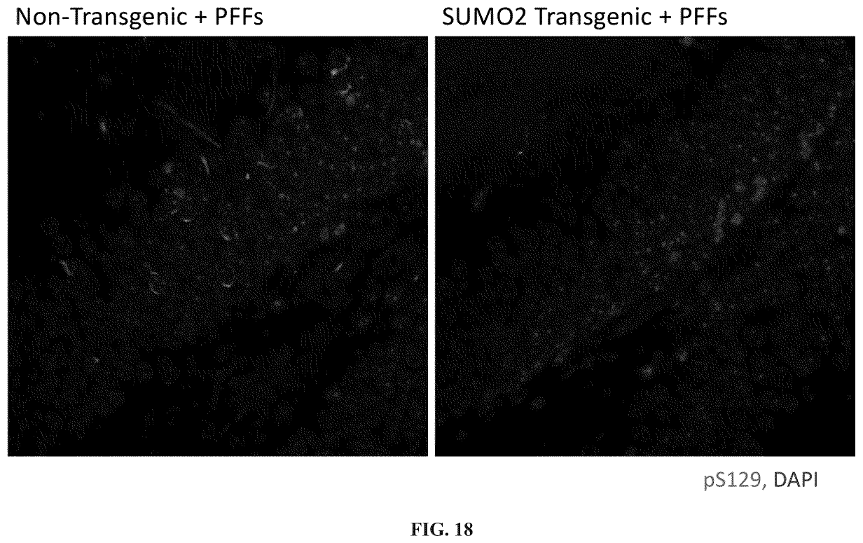

A modified version of this model was used to determine if increased expression of SUMO2 had an impact on the spreading of aSyn pathology. In this case, PFFs generated from recombinant murine aSyn were injected stereotactically into the hippocampus to generate a confined seeded area from which the pathology spreading could be monitored. PFFs were injected into wild-type control mice expressing endogenous levels of SUMO2 and a transgenic model over-expressing human SUMO2 through the use of a neuron-specific prion promoter (Fioriti, L., et al. Genetic and pharmacologic enhancement of SUMO2 conjugation prevents and reverses cognitive impairment and synaptotoxicity in a preclinical model of Alzheimer's disease. Alzheimer's and Dementia 21(2025)). (FIG. 17). The pathology was allowed to spread for 6-weeks at which time the injected hemisphere was examined by immunofluorescence. FIG. 18 provides the immunofluorescence analysis of the hippocampus region from control non-transgenic mice and SUMO2 transgenics that were inoculated with α-synuclein preformed fibrils (PFFs). Control mice displayed extensive PD-like pathology as determine by staining for the phosphorylated Ser-129 residue (pS129; green). Over-expressing SUMO2 transgenics has significantly reduced pathology suggesting that high levels of SUMO2 were able to prevent the propagation of the pathology normally induced by PFF inoculations. It was observed that the SUMO2 transgenics show similar prevention of cognitive decline and synaptic impairments in transgenic models of AD-related amyloid pathology. Therefore, it is expected that BioSenA will have comparable activity as SUMO2 over-expression and also reduce and/or halt the propagation of a-synuclein pathology. DAPI (blue) was used as a counterstain. The findings indicated that increased SUMO2 expression resulted in a decrease in the level of pS129 staining consistent with the prevention of aSyn propagation Quantification of the pathology as a measure of total area revealed that the SUMO2 transgenics reduced aS129 by approximately 4-fold in the hippocampus.

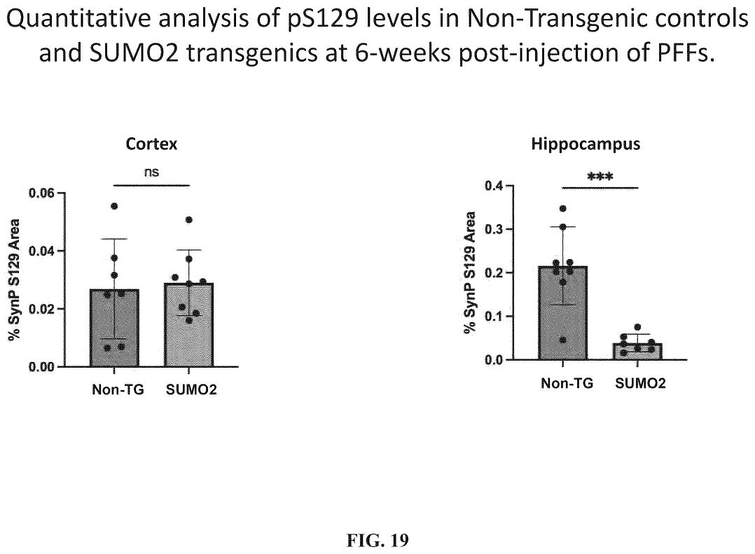

FIG. 19 provides quantitative analysis of pS129 levels in Non-Transgenic controls and SUMO2 transgenics at 6-weeks post-injection of PFFs. Synuclein pre-formed fibrils (PFFs) were injected directly into the hippocampus in Non-transgenic (Non-Tg) or SUMO2 over-expressing transgenics (SUMO2) and phosphorylated a-synuclein (pS129) was quantified by immunofluorescence at 6-weeks post-injection. Levels of pS129 in the cortex where spreading occurs at much later timepoints were not significantly different but the higher expression of SUMO2 in the transgenic animals resulted in a significant and ˜4-fold decrease in pS129 within the hippocampus (i.e., the site of PFF injection and subsequent spreading). Mean±SD, ***; P≤0.001 by Unpaired Samples t Test. Biochemical analysis of the contralateral hemisphere by immunoblotting for pS129 indicated there were no changes in the levels for the SUMO2 or wild-type mice (data not shown). This is consistent with the fact that the pathology at this early stage had not spread further than the injection site within the hippocampus. Given the comparable activities of BioSenA and SUMO2, these findings indicate that treatment with the biologic similar to that conducted in tau transgenics may reduce pathology in PD by preventing the spread of aSyn aggregates to other brain regions.

### Methods

SUMO2 and APP Transgenic Mouse Models. All mouse work was approved by the Animal Care Committee of the University of Toronto and the University Health Network in accordance with the regulations of the Canadian Council on Animal Care and the IACUC committee of Columbia University. The APP (TgCRND8) transgenic mice expressed full-length APP695 containing the Indiana and Swedish mutation within the Ab sequence also using the prion cos-tet promoter. (Chishti, M. A., et al. Early-onset amyloid deposition and cognitive deficits in transgenic mice expressing a double mutant form of amyloid precursor protein 695. J Biol Chem 276, 21562-21570 (2001)). Transgenics expressing the full-length human SUMO2 transgenic mice were generated on an FVB background and, when crossed with APP mice were maintained on a mixed 129S1/FVB background. Correspondingly, APP (129S1) were crossed to non-transgenic FVB mice to generate control mice on an identical background. Non-transgenic (Non-Tg) animals are the littermates of TgCRND8 mice (APP+/−). SUMO2+/+ APP+/− double transgenic (SUMO2-APP) mice were produced from crosses of SUMO2+/+ transgenics with APP+/− animals. Both male and female mice were examined at the 9-month endpoint under investigation.

Immunoblotting and Antibodies. Samples (5-45 μg total protein) were run on 4-12% Novex tris-glycine gels (Life Technologies) and immunoblotting was performed as previously described. (Satoh, K., Abe-Dohmae, S., Yokoyama, S., St George-Hyslop, P. & Fraser, P. E. ATP-binding cassette transporter A7 (ABCA7) loss of function alters Alzheimer amyloid processing. J Biol Chem 290, 24152-24165 (2015)). The antibodies used were: rabbit polyclonal SUMO2 (1:1000) was generated and affinity-purified using methods described previously (Matsuzaki, S., et al. SUMO1 Affects Synaptic Function, Spine Density and Memory. Scientific reports 5, 10730-10730 (2015)); rabbit monoclonal SUMO1 (1:1000; C21A7, Cell Signaling Technology); rat monoclonal SUMO2/3 (1:1000; 3H12, Millipore Sigma); rabbit polyclonal Ubc9 (1:1000; Abcam, ab33044); rabbit polyclonal PSD95 (1:1000; Abcam, ab18258); mouse monoclonal Actin (1:10,000; AC-74, Sigma-Aldrich); rabbit monoclonal GAPDH (1:10000; 14C10 Cell Signaling). Full-length APP and C-terminal fragment of APP levels of cell lysate and brain lysate were analyzed by immunoblotting using the monoclonal antibody C1/6.1. (Jiang, Y., et al. Alzheimer's-related endosome dysfunction in Down syndrome is Aβ-independent but requires APP and is reversed by BACE-1 inhibition. Proceedings of the National Academy of Sciences of the United States of America 107, 1630-1635 (2010)). After washing HRP-conjugated anti-mouse (1:3000), anti-rabbit (1:5000) or anti-goat (1:5000) was applied for 1 hour at room temperature and bands were visualized by enhanced chemiluminescence (ECL).

ELISA Quantification of Soluble/Insoluble Ab42 and APP Processing. Frozen brain hemispheres or dissected brain regions (cortex, hippocampus, cerebellum) were weighed for whole protein extraction. Tissues (100 mg) were homogenized in 500 μl of buffer containing 20 mM Tris, pH 7.4; 250 mM sucrose; 1 mM EDTA; 1 mM EGTA and EDTA-free protease inhibitors (Sigma-Aldrich) followed by sonication. Homogenates were centrifuged for 5 minutes at 27,000 g and supernatants collected. Protein quantification was performed by Bradford assay (BioRad) using the microplate format, as per the manufacturer's instructions. Readings were performed on a Spectra Max i3 (Molecular Devices). Samples were diluted in homogenization buffer to 3 or 6 mg/ml concentration and stored in an equal volume of Laemmli buffer at −80° C. Using the hippocampal, cortical and cerebellar lysates described above both soluble and insoluble forms of Ab40/42 and secreted APPb/WT were measured using commercially available ELISA kits (IBL international) as previously described². Soluble Ab40/42 is extracted from a 10% (w/v) tissue homogenate (20 mM Tris-HCl; 0.25M sucrose; 1 mM EDTA/EGTA) using an equal volume of 4% diethylamine in 100 mM NaCl. The insoluble amyloid is obtained by centrifugation (100,000 g for 1 hr) and extracted by sonication using cold formic acid. Quantification of the endogenous murine Ab40/42 was performed by ELISA (Invitrogen)². Levels of the secreted APP b-fragment (sAPPb/Sw) in the APP-Tg and SUMO2-APP transgenics was assessed using the commercially available ELISA (IBL) which was used following the manufacture's protocol.

Immunohistochemistry and Image Analysis of Amyloid Loads. Brain hemispheres were fixed in 10% formalin (Sigma-Aldrich) overnight at 4° C. then immersed in 70% ethanol. Serial sections (5 μm) of paraffin embedded tissue were stained for amyloid plaques. Amyloid deposits were identified using an HRP-conjugated primary Ab-specific antibody (6E10-HRP, Signet) and visualized with DAB following pre-treatment with 70% formic acid. Dense and diffuse plaque staining were assessed by measuring the amyloid positive area over total area. (Bachstetter, A. D., et al. Early stage drug treatment that normalizes proinflammatory cytokine production attenuates synaptic dysfunction in a mouse model that exhibits age-dependent progression of alzheimer's disease-related pathology. Journal of Neuroscience 32, 10201-10210 (2012)). Briefly, immunostained sections (5 μm) were scanned with Mirax Scan (Zeiss) and assessed using ImageScope (Aperio). Slides were scanned using the Mirax Scan v. 1.11 software and Zeiss Mirax Slide Scanner at 20× magnification with a Zeiss 20×/0.8 objective lens and a Marlin F146-C CCD camera. The rendered digital images were analyzed using the Color Deconvolution Algorithm in the Aperio Imagescope software, as described previously. (Durk, M. R., et al. 1α, 25-dihydroxyvitamin D3 reduces cerebral Amyloid-β accumulation and improves cognition in mouse models of Alzheimer's disease. Journal of Neuroscience 34, 7091-7101 (2014)). RGB values were determined for both the applied hematoxylin and DAB stains. DAB was chosen as the positive color channel for identifying and quantifying Aβ stained plaques within different areas of the brain (cortex and hippocampus). Recognition and measurement of dense and diffuse plaque areas were achieved by setting the threshold values of color intensity. The strong positive threshold was set to 80, correlating with dense staining. The medium positive threshold was set to 160, correlating with medium/diffuse staining and the weak positive threshold was set to 0. In this way, the amyloid-positive area, as well as intensity of Aβ staining, was quantified in different brain regions, allowing for the quick, objective comparison between brains from different animals.

BioSenA and BioSenI Expression and Purification. The human SUMO2 construct was inserted into the pET-28 expression plasmid using XbaI and BamHI restriction sites. BioSenA includes the full-length SUMO2 with hexa-His tag and leader sequence (HHHHHHPMSDYDIPTTENLYFQGA (SEQ ID NO: 1)) with a TEV cleavage site (GA) immediately N-terminal to the SUMO2 sequence. The inactive BioSenI contains a mutated C-terminal conjugation site where the di-glycine (GGVY (SEQ ID NO: 6)) was substituted with alanine residues (AAVY (SEQ ID NO: 7)). Proteins were expressed in E. coli grown in Terrific Broth containing antibiotic (50 μg/ml kanamycin) with induction (1 mM IPTG) at 37° C. for 4 hours. Cell pellets were resuspended in buffer (20 mM HEPES, 300 mM NaCl, 20 mM imidazole; pH 7.4) and lysed by sonication. Lysates were clarified by centrifugation (10,000 g) and applied to a nickel affinity column (Qiagen Superflow). Bound protein was washed with buffer containing 40 mM imidazole and eluted with 300 mM imidazole. Eluates were pooled and buffer exchanged by dialysis to phosphate buffered saline with 1 mM dithiothreitol. Protein was concentrated using Amicon Ultra-15 with a molecular weight cut-off of 10 kD and purified by size exclusion column (Cytvia Superdex 75) in PBS. Fractions containing purified BioSenA or BioSenI were sterile filtered with Acrodisc units with Mustang E membrane and aliquots snap frozen in liquid nitrogen then stored at −80° C.

Pharmacokinetics of BioSenA. Purified BioSenA containing a N-terminal hexa-His tag was administered to wild-type C57BL/6 mice at 20 mg/kg by subcutaneous and intravenous route of injection. Plasma and cortex samples were isolated at indicated times over a 24 hr period. BioSenA was quantified in plasma and brain using a modified His-tag ELISA (Abcam; ab128573). Brain tissues were homogenized using the Ab42 ELISA protocol and treated with 5M urea then diluted prior to loading onto the His-tag ELISA plates. Samples were incubated on the capture plates at 4° C. overnight then washed with PBS and detection was performed using a SUMO2 rabbit polyclonal antibody (dilution 1:200). Standard curves were determined with purified BioSenA.

### Electrophysiological Recordings

Electrophysiological recordings were performed as described previously. (Puzzo, D., et al. Tau is not necessary for amyloid-ß-induced synaptic and memory impairments. Journal of Clinical Investigation 130, 4831-4844 (2020)). Briefly, coronal hippocampal slices were cut by a chopper at a thickness of 400 μm and transferred to a recorded chamber where they were allowed to recover for 2 hours. During the recovery period and the recording, slices were maintained at 29° C. and perfused with artificial cerebrospinal fluid (ACSF) containing NaCl (124.0 mM), KCl (4.4 mM), Na₂HPO₄ (1.0 mM), NaHCO₃ (25.0 mM), CaCl₂) (2.0 mM), MgCl₂ (2.0 mM), and glucose (10.0 mM). ACSF was bubbled with 95% 02 and 5% CO₂ (flow rate of 2 mL/min). Field excitatory post-synaptic potentials were measured after stimulating the Schaffer collateral fibers by a bipolar tungsten electrode placed at the CA3 and recording at the stratum radiatum of CA1 with a glass pipette filled with ACSF. Baseline was recorded every minute at an intensity eliciting a response approximately 35% of the maximum evoked response. After 20-30 minutes of stable baseline, LTP was induced through a theta-burst stimulation (4 pulses at 100 Hz, with the bursts repeated at 5 Hz and 3 tetani of 10-burst trains administered at 15-second intervals). Responses were recorded for 2 hours after tetanization and measured as fEPSP slope expressed as percentage of baseline. Results were analyzed in pClamp 11 (Molecular Devices).

### Behavioral Testing

For evaluating associative fear memory, we employed the fear conditioning test, performed in two consecutive days. The first day, the animals were placed in the fear conditioning chamber (33 cm×20 cm×22 cm) (Noldus) for 2 minutes before the presentation of a discrete tone of 2880 Hz at 85 Db (conditional stimulus). In the last 2 s of the tone, mice received a foot shock of 0.8 mA intensity (unconditional stimulus). After the pairing of the 2 stimuli, mice were left in the chamber for another 30 s in the absence of a stimulus. The second day, mice were returned to the same conditioning chamber for another 5 min without the presence of tone of shock to evaluate contextual fear memory. Freezing behavior, distinguished by the absence of movement except breathing, was monitored during the test using a vision tracking and analysis system (Ethovision XT, Noldus).

### Statistical Analysis

Experiments were performed in blind. Data were expressed as means f standard error mean (SEM). For electrophysiological recordings, groups were compared by 2-way ANOVA considering 120 min of recording after tetanus. Behavioral experiments were designed in a balanced fashion and, for each condition mice were trained and tested in three to four separate sets of experiments. One-way ANOVA with Bonferroni post-hoc correction was used for comparisons among the groups of mice.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention. Those skilled in the art will recognize, or be able to ascertain, using no more than routine experimentation, numerous equivalents to the specific embodiments described specifically in this disclosure. Such equivalents are intended to be encompassed in the scope of the following claims.

### REFERENCES

1. Karran, E. & De Strooper, B. Nature Reviews Drug Discovery 21, 306-318 (2022).
2. Tzioras, M., McGeachan, R.I., Durrant, C. S. & Spires-Jones, T.L. Nature Reviews Neurology 19, 19-38 (2023).
3. Henley, J. M., et al. Journal of Neurochemistry 156, 145-161 (2021).
4. Luo, H.-B., et al. Proceedings of the National Academy of Sciences of the United States of America 111, 16586-16591 (2014).
5. Rott, R., et al. Proceedings of the National Academy of Sciences of the United States of America 114, 13176-13181 (2017).
6. Saitoh, H. & Hinchey, J. J. Biol. Chem. 275, 6252-6258 (2000).
7. Knock, E., et al. Neurobiol Dis 110, 154-165 (2018).
8. Khayachi, A., et al. Nature Communications 9(2018).
9. Matsuzaki, S., et al. Scientific reports 5, 10730-10730 (2015).
10. Lee, L., et al. Scientific reports 4, 7190-7190 (2014).
11. Wang, Z., et al. Spinal cord 51, 139-143 (2013).
12. Chishti, M. A., et al. J. Biol. Chem. 276, 21562-21570 (2001).
13. Hendriks, I. A., et al. Nature Communications 9(2018).
14. Lamoliatte, F., et al. Nature Communications 5(2014).
15. Menon, S., et al. Viral alpha-synuclein knockdown prevents spreading synucleinopathy. Brain Commun 3, fcab247 (2021).
16. Lau, A., et al. alpha-Synuclein strains target distinct brain regions and cell types. Nat Neurosci 23, 21-31 (2020).
17. Ghanem, S. S., et al. α-Synuclein phosphorylation at serine 129 occurs after initial protein deposition and inhibits seeded fibril formation and toxicity. Proceedings of the National Academy of Sciences of the United States of America 119(2022).
18. Camicioli, R., et al. Parkinson's disease is associated with hippocampal atrophy. Mov Disord 18, 784-790 (2003).
19. Villar-Conde, S., et al. The Human Hippocampus in Parkinson's Disease: An Integrative Stereological and Proteomic Study. J Parkinsons Dis 11, 1345-1365 (2021).
20. Liu, A. K. L., et al. Hippocampal CA2 Lewy pathology is associated with cholinergic degeneration in Parkinson's disease with cognitive decline. Acta Neuropathol Commun 7, 61 (2019).
21. Miki, Y., et al. Hippocampal alpha-synuclein pathology correlates with memory impairment in multiple system atrophy. Brain 143, 1798-1810 (2020).
22. Ando, T., et al. Multiple system atrophy variant with severe hippocampal pathology. Brain Pathol 32, e13002 (2022).
23. Adamowicz, D. H., et al. Hippocampal alpha-Synuclein in Dementia with Lewy Bodies Contributes to Memory Impairment and Is Consistent with Spread of Pathology. J Neurosci 37, 1675-1684 (2017).
24. Uchihara, T. & Giasson, B. I. Propagation of alpha-synuclein pathology: hypotheses, discoveries, and yet unresolved questions from experimental and human brain studies. Acta Neuropathologica 131, 49-73 (2016).
25. Polinski, N.K. A Summary of Phenotypes Observed in the In Vivo Rodent Alpha-Synuclein Preformed Fibril Model. J Parkinsons Dis (2021).
26. Fioriti, L., et al. Genetic and pharmacologic enhancement of SUMO2 conjugation prevents and reverses cognitive impairment and synaptotoxicity in a preclinical model of Alzheimer's disease. Alzheimer's and Dementia 21(2025).

What is claimed is:

1. A method for treating Alzheimer's Disease in a subject, the method comprising administering to a subject a therapeutic amount of a pharmaceutical composition comprising a recombinant small ubiquitin modifier 2 (SUMO2) analogue.

2. A method for increasing memory retention in a subject afflicted with a neurodegenerative disease, the method comprising administering to a subject a therapeutic amount of a pharmaceutical composition comprising recombinant SUMO2 analogue.

3. A method for increasing synaptic plasticity in a subject afflicted with a neurodegenerative disease, the method comprising administering to a subject a therapeutic amount of a pharmaceutical composition comprising recombinant SUMO2 analogue.

4. The method of claim 3, wherein synaptic plasticity comprises learning, memory, or a combination thereof.

5. The method of claim 3, wherein synaptic plasticity comprises long term potentiation (LTP).

6. A method for preventing Alzheimer's Disease in a subject, the method comprising administering to a subject a therapeutic amount of a pharmaceutical composition comprising a recombinant SUMO2 analogue.

7. The method of claim 1, wherein the recombinant SUMO2 analogue comprises a sequence that is at least 70% identical to a sequence of the SUMO2.

8. The method of claim 1, wherein the recombinant SUMO2 analogue comprises a N-terminal tag and a leader sequence N-terminal to the SUMO2 sequence.

9. The method of claim 1, wherein the recombinant SUMO2 analogue comprises a SUMO2 sequence with a hexa-His tag and a leader sequence (HHHHHHPMSDYDIHTTENLYFQGA) with a TEV cleavage site (GA) immediately N-terminal to SUMO2 sequence.

10. The method of claim 1, wherein the recombinant SUMO2 analogue has the sequence
HHHHHHPMSDYDIPTTENLYFQGANDHINLKVAGQDGSVVQFKIKRHTP
LSKLMKAYCERQGLSMRQIRFRFDGQPINETDTPAQLEMEDEDTIDVEQ
QQTGGVY.

11. A pharmaceutical composition comprising a recombinant SUMO2 analogue.

12. The pharmaceutical composition of claim 11, wherein the recombinant SUMO2 analogue comprises a sequence that is at least 70% identical to a sequence of the SUMO2.

13. The pharmaceutical composition of claim 11, wherein the recombinant SUMO2 analogue comprises a hexa-His tag and a leader sequence (HHHHHHPMSDYDIPTTENLYFQGA) with a TEV cleavage site (GA) immediately N-terminal to SUMO2 sequence.

14. The pharmaceutical composition of claim 11 wherein the recombinant SUMO2 analogue has the sequence
HHHHHHPMSDYDIPTTENLYFQGANDHINLKVAGQDGSVVQFKIKRHTP
LSKLMKAYCERQGLSMRQIRFRFDGQPINETDTPAQLEMEDEDTIDVFQ
QQTGGVY.
